SEQUENCE LISTING (9 sequences)

SEQ ID NO: 1 - protein, 20 aa, Artificial Sequence
  BMP-2 peptide
KIPKASSVPTELSAISTLYL

SEQ ID NO: 2 - protein, 14 aa, Artificial Sequence
  QK peptide
KLTWQELQLKYKGI

SEQ ID NO: 3 - protein, 17 aa, Artificial Sequence
  QK peptide
KLTWQELQLKYKGIGGG

SEQ ID NO: 4 - protein, 24 aa, Artificial Sequence
  QK peptide
KLTWQELQLKYKGIGGGEEEEEEE

SEQ ID NO: 5 - protein, 12 aa, Artificial Sequence
  PR1P peptide
DRVQRQTTTVVA

SEQ ID NO: 6 - protein, 40 aa, Artificial Sequence
  Lv peptide
DSLLAVRWFFAHSFDSQEALMVKMTKLRVVQYYGNFSRSA

SEQ ID NO: 7 - protein, 12 aa, Artificial Sequence
  RoY peptide
YPHIDSLGHWRR

SEQ ID NO: 8 - protein, 10 aa, Artificial Sequence
  periostin peptide
WDNLDSDIRR

SEQ ID NO: 9 - protein, 16 aa, Artificial Sequence
  periostin peptide
APSNEAWDNLDSDIRR

(19) United States
(12) Patent Application Publication
(10) Pub. No.: US 2023/0093766 A1
(43) Pub. Date: Mar. 23, 2023

(54) COMPOSITIONS AND METHODS FOR COATING BONE GRAFTS

(71) Applicants: BOARD OF REGENTS OF THE UNIVERSITY OF NEBRASKA, Lincoln, NE (US); UNIVERSITY OF ROCHESTER, ROCHESTER, NY (US)

(72) Inventors: Jingwei XIE, Omaha, NE (US); Xinping ZHANG, Pittsford, NY (US)

(21) Appl. No.: 17/795,604

(22) PCT Filed: Feb. 8, 2021
(86) PCT No.: PCT/US21/17011

§ 371 Date: Jul. 27, 2022

Related U.S. Application Data
(60) Provisional application No. 62/971,247, filed on Feb. 7, 2020.

(51) Int. Cl. A61L 27/36; A61L 27/54; A61L 27/34; A61K 38/18; B05D 1/04

(52) U.S. Cl. CPC A61L 27/3608; A61L 27/54; A61L 27/34; A61K 38/1841; A61L 27/365; B05D 1/04; A61L 2300/414; A61L 2300/232; A61L 2420/02; A61L 2420/06; A61L 2420/08

(57) ABSTRACT

Coated bone grafts are provided as well as methods of use thereof and methods of making. In accordance with the instant invention, methods of preparing a coated bone graft (e.g., bone allograft) are provided. In certain embodiments, the method comprises electrospraying a composition comprising a polymer and, optionally, an agent, particularly a therapeutic agent, onto the surface of the bone graft. Therapeutic agents include, without limitation: bone stimulating agents, anti-fibrotic agents, antimicrobials, anti-inflammatory agents, and pro-angiogenesis agents.

This application claims priority under 35 U.S.C. § 119(e) to U.S. Provisional Patent Application No. 62/971,247, filed Feb. 7, 2020. The foregoing application is incorporated by reference herein.

### FIELD OF THE INVENTION

This application relates to the fields of bone grafts. More specifically, this invention provides coated bone grafts, compositions and methods of synthesizing the coated bone grafts, and methods of use thereof.

### BACKGROUND OF THE INVENTION

Several publications and patent documents are cited throughout the specification in order to describe the state of the art to which this invention pertains. Each of these citations is incorporated herein by reference as though set forth in full.

Bone grafts and substitutes are widely used in orthopedic surgeries for various repair and reconstruction applications. More than 500,000 bone grafting procedures are performed in the United States annually and 2.2 million worldwide with an estimated market size valued at 2.4 billion dollars in 2016 (Greenwald, et al. (2001) J. Bone Joint Surg. Am., 83-A Suppl 2(2):98-103; Grand View Research, I. Bone Grafts and Substitutes Market Size, Share & Trends Analysis Report By Material Type (Natural, Synthetic), Market Watch, 2018). Although autograft is the gold standard for bone graft transplant, limited resource, donor site pain, morbidity, and complications have greatly restricted its usage. Allograft remains as a top choice for revision surgery due to its immediate availability, superior mechanical property and absent donor site morbidity. However, allograft transplantation is reported to have 60%, 10-year post-implantation failure rate due to fibrotic nonunions, infections, and secondary fractures (Goldberg, et al. (1987) Clin. Orthop., 1987:7-16; Wheeler, et al. (2005) Clin. Orthop. Relat. Res., 435:36-42). To improve healing and osseointegration, strategies have been developed to enhance the osteogenic and angiogenic properties of the structural allografts, including coating bone allografts with bioactive molecules, viral vectors, and osteogenic cells (Petrie Aronin, et al. (2010) Biomaterials 31:6417-24; Koefoed, et al. (2005) Mol. Ther., 12:212-8; Hoffman, et al. (2013) Biomaterials 34:8887-98; Wang, et al. (2018) Biomaterials 182:279-88). While some success has been achieved, these strategies are associated with problems such as i) fibrosis tissue formation, ii) uneven callus formation, iii) safety of virus vectors, and iv) challenges in production of transplantable cells. These existing problems significantly hamper the successful translation of the modified allografts to the clinics. In view of the current lack of bone graft materials that can match the mechanical performance of an allograft, new methodologies aimed at enhancing the biological performance of the structural allograft for reconstruction of bone defects are urgently needed.

### SUMMARY OF THE INVENTION

In accordance with the instant invention, methods of preparing a coated bone graft (e.g., bone allograft) are provided. In certain embodiments, the method comprises electrospraying a composition comprising a polymer and, optionally, an agent, particularly a therapeutic agent, onto the surface of the bone graft. Therapeutic agents include, without limitation: bone stimulating agents, anti-fibrotic agents, antimicrobials, anti-inflammatory agents, and pro-angiogenesis agents. In certain embodiments, the therapeutic agent is a bone stimulating agent such as a bone morphogenetic protein (e.g. bone morphogenetic protein 2 (BMP-2) or a fragment thereof). In certain embodiments, the polymer is a hydrophobic polymer. In certain embodiments, the polymer is poly(lactide-co-glycolide). In certain embodiments, the composition comprises a bone stimulating agent such as bone morphogenetic protein 2 (BMP-2) or a fragment thereof and an anti-fibrotic agent such as corilagen. The methods of the instant invention may comprise repeating the electrospraying with different compositions to generate a multiple layer coating. In certain embodiments, the method comprises electrospraying a first composition comprising a polymer and, optionally, a therapeutic agent onto the surface of the bone graft, and ii) electrospraying a second composition comprising a polymer and, optionally, a therapeutic agent onto the surface of the coating produced by step i). In certain embodiments, the coating of the coated bone graft is about 1 μm to about 1 mm thick. The methods of the instant invention may further comprise freeze drying and/or lyophilizing the synthesized coated bone graft and/or mineralizing the synthesized coated bone graft. The instant invention also encompasses the coated bone grafts synthesized by these methods.

In accordance with another aspect of the instant invention, coated bone grafts are provided. The coated bone grafts comprise a bone graft and an electrosprayed coating on the surface of the bone graft, wherein the electrosprayed coating comprises a polymer and, optionally, an agent, particularly a therapeutic agent. Examples of therapeutic agent include, without limitation: bone stimulating agents, anti-fibrotic agents, antimicrobials, anti-inflammatory agents, and pro-angiogenesis agents. In certain embodiments, the therapeutic agent is a bone stimulating agent such as a bone morphogenetic protein (e.g., bone morphogenetic protein 2 (BMP-2) or a fragment thereof). In certain embodiments, the polymer is a hydrophobic polymer. In certain embodiments, the polymer is poly(lactide-co-glycolide). In certain embodiments, the coating comprises a bone stimulating agent such as bone morphogenetic protein 2 (BMP-2) or a fragment thereof and an anti-fibrotic agent such as corilagen. The coated bone grafts of the instant invention may comprise more than one layer or coating. In certain embodiments, the coating of the coated bone graft is about 1 μm to about 1 mm thick.

In accordance with another aspect of the instant invention, methods for treating a bone defect in a subject are provided. The methods comprise implanting the coated bone graft of the instant invention into the subject, particularly at the site of the bone defect. In certain embodiments, the bone graft is a bone allograft.

### BRIEF DESCRIPTION OF THE DRAWINGS

FIG. 1A provides a schematic illustrating the fabrication of engineered bone allografts using electrospray deposition. FIG. 1B provides a schematic illustration of implantation of the polymer-coated allografts to repair as segmental femoral bone defect.

FIG. 2A provides scanning electron microscopy (SEM) images of half of a bone allograft uniformly coated with BMP-2 peptide-loaded poly(lactide-co-glycolide) (PLGA). FIG. 2B provides a high magnified cross-section image of the coated allograft illustrating the thickness of PLGA coating. FIG. 2C provides the half of allograft coated with BMP-2 peptide-loaded PLGA imaged with Profilm3D® filmetrics system. FIG. 2D provides a graph of BMP-2 loadings and in vitro release profiles from engineered bone allografts: comparison of PCL-gelatin (triangles) and PLGA coatings (circles).

FIG. 3A provides images of the examination of healing in each group by X-ray at weeks 0, 3, and 5 post-surgery. FIG. 3B provides a microCT and histologic analyses of allograft healing at week 5 post-surgery. Compared with allograft PLGA control, BMP-2 peptide coated allograft induced new bone formation along the surface of allograft (arrows in FIG. 3B). MicroCT quantification of new bone volume at donor side and host side callus are also provided (n=6). FIG. 3C provides an illustration of new bone overlaid on allograft surface. MicroCT quantification of new bone volume at donor side (FIG. 3D) and host side callus (FIG. 3E). FIG. 3F provides a quantification of percent allograft surface area overlaid by new bone (n=6).

FIGS. 4A-4C provide hematoxylin and eosin (H&E) alcian blue staining of the sections from allograft (FIG. 4A), PLGA allograft (FIG. 4B), and BMP-2 peptide allograft (FIG. 4C). FIGS. 4D-4F provide quantitative histomorphometric analyses (n=6) of the percentage of bone, cartilage and fibrotic tissue area at donor side (FIG. 4D), host side (FIG. 4E), and total callus (FIG. 4F).

FIGS. 5A and 5B shows torsional biomechanical testing in grafted femurs at weeks 7 post-surgery. Ultimate torque (FIG. 5A) and torsional rigidity (FIG. 5B) in comparison with normal bone are illustrated. n=3, p<0.05.

FIG. 6A provides an image of H&E staining at the host-graft interface. FIGS. 6B-6D provide immunofluorescence images of pSMAD3 in fibrotic tissue (FIG. 6B), pSMAD5 in fibrotic tissue (FIG. 6C) and bone/cartilage (FIG. 6D) at the cortical bone junctions. FIGS. 6E-6G provide representative microscopic images of H&E staining and pSMAD3 staining in allograft (FIG. 6E), allograft coated with PLGA (FIG. 6F) and allograft coated with BMP-2 peptide loaded PLGA (FIG. 6G) at indicated magnification.

FIG. 7 provides graphs of alkaline phosphatase (ALP) and osterix (Osx) expression in periosteal progenitor cells isolated from autograft periosteum after 10 days untreated (ctrl) or treated with BMP-2, corilagin, or both.

### DETAILED DESCRIPTION OF THE INVENTION

Surface modification of biomaterials have been used to improve the biological interaction and integration between implant and host tissue. Currently available technologies for surface modification of bone substitutes not only allow alterations of surface morphology and biochemistry of the implant, but also enable incorporation of bioactive molecule into the surface layer of the implants through coating. By creating a highly porous cortical bone surface using a mixture of poly(propylene fumarate) and hydroxylapatite, the modified allograft surface can promote the migration and proliferation of the osteoblasts adjacent to bone surface (Bondre, et al. (2000) Tissue Eng., 6:217-27; Lewandrowski, et al. (2002) Tissue Eng., 8:1017-27). Using a classic polymer coating technique—dipping and rapid drying—a number of growth factors and bioactive molecules has been delivered to an allograft site to enhance allograft healing and incorporation (Petrie Aronin, et al. (2010) Biomaterials 31:6417-24; Sharmin, et al. (2015) J. Biomed. Mater Res. A, 103:2847-54; Sharmin, et al. (2017) J. Orthop. Res., 35:1086-95; Sharmin, et al. (2019) J. Biomed. Mater. Res. B Appl. Biomater., 107:1002-10). In addition, functionalization of allograft surface through binding of hydroxyapatite to osteoinductive peptides has been shown beneficial effects to enhance healing of allograft bone (Culpepper, et al. (2013) Biomaterials 34:1506-13; Culpepper, et al. (2013) Biomaterials 34:2455-62; Culpepper, et al. (2014) J. Biomed. Mater. Res. A, 102:1008-16). While these methods have demonstrated potential for clinical translation of modified allograft, the control of drug loading and distribution throughout the allograft surface has been problematic. Novel methods to enhance the bio-distribution, reproducibility, and versatility of the coating is needed.

Electrospraying is an efficient method to produce uniformly distributed and dispersed droplets/particles on various surfaces ranging from nanometers to micrometers (Xie, et al. (2015) Chem. Engr. Sci., 125:32-57). This technique utilizes high electric voltage to disintegrate or atomize the bulk liquid jet into fine liquid droplets of identical charge for surface coating. The basic experimental set up of electrospray is similar to that used in electrospinning. It generally comprises a high voltage power supply, a syringe pump, and a plastic or glass syringe capped by a metallic needle with defined diameter and a grounded collector or substrate for collecting the particles (Xie, et al. (2015) Chem. Engr. Sci., 125:32-57; Khan, et al. (2017) Food Eng. Rev., 9:112-119). Electrospray deposition has been used to fabricate various biodegradable films/coatings for sustained drug delivery or coating for stent applications (Xie, et al. (2015) Chem. Engr. Sci., 125:32-57; Boda, et al. (2018) J. Aerosol. Sci., 125:164-81). The control of coating topography on implant devices via varying polymer concentration, viscosity and voltage can be used to regulate their integration with the surrounding tissue. Thus, electrospray can be advantageously applied for encapsulation and delivery of a broad range of drug carriers including liposomes, dendrimers, polymeric micelles, and/or therapeutic drugs (Sridhar, et al. (2013) Biomatter., 3(3):e24281). Compared with other existing coating techniques such as dip-coating, physical adsorption, and growth factor conjugation (Sharmin, et al. (2015) J. Biomed. Mater Res. A, 103:2847-54; Sharmin, et al. (2017) J. Orthop. Res., 35:1086-95; Sharmin, et al. (2019) J. Biomed. Mater. Res. B Appl. Biomater., 107:1002-10), polymer-mediated electrospray deposition will provide the following unique features: i) high efficiency; ii) uniform coatings; iii) ease of incorporation of multiple therapeutic agents via layer-by-layer deposition; iv) elimination of cracks associated with other coating methods; v) better control of coating thickness as compared to allograft dip coating; and vi) ease to scale-up the coating process for mass production.

Herein, compositions and methods for polymer-mediated electrospray for surface modification of bone allografts (e.g., structural bone allografts) are provided. Electrospray mediated polymer deposition allows coating of bioactive molecules with well-controlled composition and structure on the surface of bone allograft. The osteogenic inductive BMP-2 peptide was uniformly coated onto allograft surface via electrospray deposition. Upon transplantation, the peptide-releasing allografts directly induced bone formation on the surface of the allografts, resulting in enhanced repair and reconstruction of bone allografts as evidenced by MicroCT and histomorphometric analyses. Thus, an off-the-shelf, versatile, and multifunctional structural bone allograft system has been provided for repair and reconstruction of bone defects (e.g., large bone defects).

Structural allografts remain a top choice for repair and reconstruction of large defects that require immediate support. Herein, a novel methodology is provided that enables coating of bioactive molecules with well-controlled composition and structure on the surface of bone allograft via polymer-mediated electrospray deposition. The coating can be easily tailored by using a variety of biomaterials to achieve desired thickness, cargo loading, and release. To evaluate the biologic effects of the coated allografts, PLGA copolymer containing BMP2 peptide were used to coat bone allografts via electrospray. The coated allografts were used to repair a 4 mm segmental defect created in mouse femur. Compared with non-coated allografts, PLGA coated allograft demonstrated inferior healing with significantly increased fibrotic tissue formation at the site of repair. In contrast, BMP-2 peptide-coated allografts demonstrated significantly improved healing as evidenced by enhanced new bone formation on the surface of allografts. With increased bone formation, the percent area of fibrotic tissue in callus was reduced in BMP-2 treated group, indicating an antagonism between osteogenesis and fibrosis. Further immunohistochemical staining demonstrated that PLGA coating significantly increased pSMAD3 level in fibrotic tissues adjacent to bone whereas coating BMP-2 peptide suppressed pSMAD3, indicating a role of TGF-β signaling in PLGA-induced fibrotic tissue formation. Taken together, the data indicate that improved coating of biological factors on allograft surface can be achieved via polymer mediated electrospray deposition with versatility and high efficiency. Modified allografts with pro-osteogenesis and anti-fibrotic properties will lead to enhanced structural bone allograft repair and reconstruction. Thus, electrospray deposition has been established as a platform technology for surface modification and coating of structural bone allografts to restore the missing osteogenic function of periosteum in repair and reconstruction of bone defects.

In accordance with the instant invention, coating bone grafts—such as bone allografts and bone autografts—are provided along with compositions and methods for their synthesis. While the instant application encompasses bone allografts, bone autografts, or any type of bone graft (e.g., xenograft), the specification will generally refer to bone allografts for simplicity while still encompassing the use of all types of bone grafts. Generally, bone grafting is the transplanting of bone tissue or bone fragment. Bone grafting may be used to repair, rebuild, and/or replace damaged, defective, diseased, and/or missing bone. Bone grafting may also be used to promote bone growth such as around a medical or dental implant or implanted device or joint.

In a particular embodiment, the coating is applied to the bone allograft by electrospraying (e.g., with solution comprising a polymer and, optionally, one or more additional agents or compounds). In a particular embodiment, the bone graft is rotated during the electrospraying. As explained hereinabove, electrospraying is a liquid atomization-based technique which produces micro/nanoparticular droplets. Electrospraying, also known as electrodynamic spraying, produces droplets of submicron sizes by means of an electric field. A common setup for electrospraying comprises a high-voltage power supply, a plastic/glass syringe capped by a metallic capillary/nozzle/needle to hold a polymer solution, a syringe pump to control the flow of the solutions, and a grounded collector. Generally, an electric potential is established between the source of the droplets and the substrate onto which the droplets are projected wherein the electric potential can exert a force on the mixture, thereby resulting in the formation of droplets from the mixture. For example, upon application of a voltage to the nozzle, a charged liquid jet will break up into droplets, forming small particles with generally narrow size distribution on the collector.

The size and morphology of electrosprayed particles and the characteristics of the electrosprayed coating can be varied by factors such as, without limitation, polymer concertation and/or molecular weight, solvent, flow rate, electric potential difference, voltage, needle gauge, flow rate, and distance between the tip of the nozzle and the bone graft and/or collector (e.g., aluminum foil collector). In a particular embodiment, the current voltage is between about 4 kV and about 14 kV, about 4 kV and about 10 kV, or about 6 kV and about 8 kV.

As stated hereinabove, the instant invention encompasses methods of coating a bone allograft comprising electrospraying the coating (e.g., a polymer solution) on to the bone allograft. The electrosprayed coating may cover at least a portion of or all or nearly all (e.g., at least 97% or at least 99% of the surface area) of the bone allograft. For example, the electrosprayed coating may cover at least 5%, 10%, 15%, 20%, 25%, 30%, 35%, 40%, 45%, 50%, 55%, 60%, 65%, 70%, 75%, 80%, 85%, 90%, 95%, 97%, or 99% of the surface of the bone allograft. In a particular embodiment, the electrosprayed coating covers at least a portion of or all or nearly all (e.g., at least 97% or at least 99% of the surface area) of the exposed surfaces of the bone allograft after transplantation. For example, the electrosprayed coating may cover at least 5%, 10%, 15%, 20%, 25%, 30%, 35%0, 40%, 45%, 50%, 55%, 60%, 65%, 70%, 75%, 80%, 85%, 90%, 95%, 97%, or 99% of the exposed surface of the bone allograft after transplantation. In a particular embodiment, the coating is electrosprayed onto a defect of the bone allograft and/or the electrosprayed coating covers at least a defect of the bone allograft.

The electrosprayed coating may be of any thickness. The electrosprayed coating may have the same or about the same thickness across the bone allograft or the thickness may vary across the bone allograft. Generally, the electrosprayed coating will have about the same thickness across the bone allograft (e.g., +/−about 5% or +/−about 10% change in thickness). The thickness of the coating may be varied, for example, by the duration of the electrospraying and/or varying the distance from the bone allograft and the nozzle during electrospraying. In a particular embodiment, the coating has a thickness from about 0.1 μm to about 1 mm, particularly about 0.1 μm to about 1 mm, about 1 μm to about 750 μm, about 1 μm to about 500 μm, about 20 μm to about 500 μm, about 50 μm to about 250 μm, or about 100 μm. In a particular embodiment, the thickness of the electrosprayed coating is less than about 3 mm, particularly less than 1 mm, less than 900 μm, less than 800 μm, less than 750 μm, less than 700 μm, less than 600 μm, less than 500 μm, less than 400 μm, less than 300 μm, less than 250 μm, less than 200 μm, less than 150 μm, less than 100 μm, or less than 50 μm. In a particular embodiment, the thickness of the electrosprayed coating is more than about 0.1 μm, particularly more than 0.5 μm, more than 1 μm, more than 5 μm, more than 10 μm, more than 20 μm, more than 25 μm, more than 30 μm, more than 40 μm, more than 50 μm, more than 60 μm, more than 70 μm, more than 75 μm, more than 80 μm, more than 90 μm, more than 100 μm, or more than 150 μm.

As explained above, a polymeric solution is electrosprayed onto the bone allograft. The polymeric solution and/or the coating on the bone allograft may comprise one or more polymers. The polymeric solution of the instant invention may comprise any polymer(s). In a particular embodiment, the polymer is biocompatible. The polymer may be biodegradable or non-biodegradable. In a particular embodiment, the polymer is FDA approved. The polymers of the instant invention may by hydrophobic, hydrophilic, amphiphilic, or mixtures thereof. In a particular embodiment, the polymer comprises a hydrophobic polymer. In a particular embodiment, the polymer comprises a hydrophilic polymer. The polymers may be, for example, a homopolymer, random copolymer, blended polymer, copolymer, or a block copolymer. Block copolymers are most simply defined as conjugates of at least two different polymer segments or blocks. The polymer may be, for example, linear, star-like, graft, branched, dendrimer based, or hyper-branched (e.g., at least two points of branching). The polymer of the invention may have, for example, from about 2 to about 10,000, about 2 to about 1000, about 2 to about 500, about 2 to about 250, or about 2 to about 100 repeating units or monomers. The polymers of the instant invention may comprise capping termini.

Examples of hydrophobic polymers include, without limitation: poly(hydroxyethyl methacrylate), poly(N-isopropyl acrylamide), poly(lactic acid) (PLA (or PDLA)), poly(lactide-co-glycolide) or poly(lactic-co-glycolic acid) (PLGA), polyglycolide or polyglycolic acid (PGA), polycaprolactone (PCL), poly(aspartic acid), polyoxazolines (e.g., butyl, propyl, pentyl, nonyl, or phenyl poly(2-oxazolines)), polyoxypropylene, poly(glutamic acid), poly(propylene fumarate) (PPF), poly(trimethylene carbonate), polycyanoacrylate, polyurethane, polyorthoesters (POE), polyanhydride, polyester, poly(propylene oxide), poly(caprolactonefumarate), poly(1,2-butylene oxide), poly(n-butylene oxide), poly(ethyleneimine), poly(tetrahydrofurane), ethyl cellulose, polydipyrolle/dicabazole, starch, polyvinylidene fluoride (PVDF), polytetrafluoroethylene (PTFE), polydioxanone (PDO), polyether poly(urethane urea) (PEUU), cellulose acetate, polypropylene (PP), polyethylene terephthalate (PET), nylon (e.g., nylon 6), polycaprolactam, PLA/PCL, poly(3-hydroxybutyrate-co-3-hydroxyvalerate) (PHBV), PCL/calcium carbonate, and/or poly(styrene).

Examples of hydrophilic polymers include, without limitation: polyvinyl alcohol (PVA), polyvinylpyrrolidone (PVP), poly(ethylene glycol) and poly(ethylene oxide) (PEO), chitosan, collagen, chondroitin sulfate, sodium alginate, gelatin, elastin, hyaluronic acid, silk fibroin, sodium alginate/PEO, silk/PEO, silk fibroin/chitosan, hyaluronic acid/gelatin, collagen/chitosan, chondroitin sulfate/collagen, and chitosan/PEO.

Amphiphilic copolymers or polymer composites may comprise a hydrophilic polymer (e.g., segment/block) and a hydrophobic polymer (e.g., segment/block) from those listed above (e.g., gelatin/polyvinyl alcohol (PVA), PCL/collagen, chitosan/PVA, gelatin/elastin/PLGA, PDO/elastin, PHBV/collagen, PLA/hyaluronic acid, PLGA/hyaluronic acid, PCL/hyaluronic acid, PCL/collagen/hyaluronic acid, gelatin/siloxane, PLLA/MWNTs/hyaluronic acid). In a particular embodiment, the amphiphilic block copolymer is an amphiphilic block copolymer comprising hydrophilic poly(ethylene oxide) (PEO) and hydrophobic poly(propylene oxide) (PPO). In a particular embodiment, the polymer is a poloxamer or an amphiphilic triblock copolymer comprising a central hydrophobic PPO block flanked by two hydrophilic PEO blocks (i.e., an A-B-A triblock structure). In a particular embodiment, the amphiphilic block copolymer is selected from the group consisting of Pluronic® L31, L35, F38, L42, L44, L61, L62, L63, L64, P65, F68, L72, P75, F77, L81, P84, P85, F87, F88, L92, F98, L101, P103, P104, P105, F108, L121, L122, L123, F127, 10R5, 10R8, 12R3, 17R1, 17R4, 17R8, 22R4, 25R1, 25R2, 25R4, 25R5, 25R8, 31R1, 31R2, and 31R4. In a particular embodiment, the polymer comprises poloxamer 407 (Pluronic® F127).

Examples of polymers particularly useful for electrospinning or electrospraying are also provided in Xie et al. (Macromol. Rapid Commun. (2008) 29:1775-1792; incorporated by reference herein; see e.g., Table 1). In a particular embodiment, examples of polymers for use in the instant invention include, without limitation: natural polymers (e.g., chitosan, gelatin, collagen type I, II, and/or III, elastin, hyaluronic acid, cellulose, silk fibroin, phospholipids (Lecithin), fibrinogen, hemoglobin, fibrous calf thymus Na-DNA, virus M13 viruses), synthetic polymers (e.g., PLGA, PLA, PCL, PHBV, PDO, PGA, PLCL, PLLA-DLA, PEUU, cellulose acetate, PEG-b-PLA, EVOH, PVA, PEO, PVP), blended (e.g., PLA/PCL, gelatin/PVA, PCL/gelatin, PCL/collagen, sodium aliginate/PEO, chitosan/PEO, Chitosan/PVA, gelatin/elastin/PLGA, silk/PEO, silk fibroin/chitosan, PDO/elastin, PHBV/collagen, hyaluronic acid/gelatin, collagen/chondroitin sulfate, collagen/chitosan), and composites (e.g., PDLA/HA, PCL/CaCO₃, PCL/HA, PLLA/HA, gelatin/HA, PCL/collagen/HA, collagen/HA, gelatin/siloxane, PLLA/MWNTs/HA, PLGA/HA). In a particular embodiment, the polymer comprises polymethacrylate, poly vinyl phenol, polyvinylchloride, cellulose, polyvinyl alcohol, polyacrylamide, PLGA, collagen, polycaprolactone, polyurethanes, polyvinyl fluoride, polyamide, silk, nylon, polybennzimidazole, polycarbonate, polyacrylonitrile, polyvinyl alcohol, polylactic acid, polyethylene-co-vinyl acetate, polyethylene oxide, polyaniline, polystyrene, polyvinylcarbazole, polyethylene terephthalate, polyacrylic acid-polypyrene methanol, poly(2-hydroxyethyl methacrylate), polyether imide, polyethylene glycol, poly(ethylene-co-vinyl alcohol), polyacrylnitrile, polyvinyl pyrrolidone, polymetha-phenylene isophthalamide, gelatin, chitosan, starch, pectin, cellulose, methylcellulose, sodium polyacrylate, starch-acrylonitrile co-polymers, and/or combinations of two or more polymers.

In a particular embodiment, the polymeric solution comprises poly(lactide-co-glycolide) copolymer (PLGA). In a particular embodiment, the polymer is PLGA (50:50). The ratio of the lactide and glycolide monomers can be varied. Such variations can tailor the degradation rate of the polymer coating. In a particular embodiment, the ratio of the lactide and glycolide monomers within PLGA is from about 10:90 to about 90:10, particularly about 20:80 to about 80:20, about 30:70 to about 70:30, about 40:60 to about 60:40, about 45:55 to about 55:45, or about 50:50.

In a particular embodiment, the instant invention encompasses electrospraying more than one polymeric solution onto the bone allograft. In other words, the instant invention encompasses bone allografts comprising more than one coating (e.g., a multi-layered coating). In a particular embodiment, the method comprises electrospraying a first polymeric solution comprising a first polymer and, optionally, one or more first additional agents or compounds and electrospraying a second polymeric solution comprising a second polymer and, optionally, one or more second additional agents or compounds. The instant invention also encompasses electrospraying a third polymeric solution, a fourth polymeric solution, a fifth polymeric solution and/or more. In a particular embodiment, at least a portion of or all of the second polymeric solution is electrosprayed onto the coating created by the electrospraying of the first polymeric solution. In a particular embodiment, a portion of or all of the second polymeric solution is electrosprayed onto a portion of the bone allograft not coated by the electrospraying of the first polymeric solution. In a particular embodiment, the second polymeric solution is electrosprayed onto the coating created by the electrospraying of the first polymeric solution onto the bone allograft, thereby generating a multilayered coating.

The various layers of a multilayered coating may have different polymer compositions compared to each other and/or comprise different additional agents or compounds. For example, a multilayered coating may comprise at least two coatings wherein the first and second coatings have the same polymer (e.g., the first and second polymer are the same) but have different additional agents or compounds (e.g., the first and second additional agent or compound are different). As another example, a multilayered coating may comprise at least two coatings wherein the first and second coatings have a different polymer(s) (e.g., the first and second polymer(s) are different) but have the same additional agents or compounds (e.g., the first and second additional agent or compound are the same).

The presence of a multilayered coating allows for the delivery of compounds at different times. For example, an agent or compound to be released first can be electrosprayed last so that it is within the outer layer. Similarly, an agent or compound to be released last can be electrosprayed first so that it is within the inner layer. By varying the distance from the surface of the coated bone allograft, the timing of the release of the additional agent or compound can be varied. Thus, the multilayered coatings of the instant invention allow for simultaneous delivery (e.g., when the agent or compound is in the same coating), delayed delivery (e.g., when the agent or compound is in an inner coating (wherein an outer coating contains an additional agent or compound or only contains polymer)), and/or sequential delivery (e.g., when one agent or compound is in an inner coating and a different agent or compound is in an outer coating).

As stated hereinabove, the polymeric solutions and/or coatings may comprise at least one additional agent or compound. Typically, the agent or compound is contained within the polymeric solution and contained or encapsulated within the polymeric coating after synthesis. The agent or compound can be present in the polymeric solution at any concentration. Generally, the polymeric solution will comprise more polymer than agent or compound. In a particular embodiment, the weight ratio of polymer to agent or compound (e.g., peptide) is from about 1:1 to about 1000:1, about 5:1 to about 500:1, about 10:1 to about 100:1, about 25:1 to about 75:1, or about 50:1.

The instant invention also encompasses bone allografts wherein the additional agent or compound is attached to the surface of the polymeric coating. For example, the additional agent or compound may be conjugated (e.g., directly or via a linker) to the polymer and/or polymeric coating. In a particular embodiment, the additional agent or compound is conjugated or linked to the polymer and/or polymeric coating (e.g., surface conjugation or coating).

Generally, the linker is a chemical moiety comprising a covalent bond or a chain of atoms that covalently attaches the ligand to the polymer or surfactant. The linker can be linked to any synthetically feasible position of the agent or compound and the polymer. Exemplary linkers may comprise at least one optionally substituted; saturated or unsaturated; linear, branched or cyclic aliphatic group, an alkyl group, or an optionally substituted aryl group. The linker may be a lower alkyl or aliphatic. The linker may also be a polypeptide (e.g., from about 1 to about 10 amino acids, particularly about 1 to about 5). The linker may be non-biodegradable and may be a covalent bond or any other chemical structure which cannot be substantially cleaved or cleaved at all under physiological environments or conditions. In a particular embodiment, the linker is biodegradable.

In a particular embodiment, the additional agent or compound is administered separately from the bone allograft (e.g., in a composition with a pharmaceutically acceptable carrier). The separately administered agent or compound may or may not also be incorporated into the bone allografts. For examples, the additional agent or compound may be administered in a (separate) composition (e.g., comprising a pharmaceutical carrier) from the bone allograft. The additional agent or compound may be administered simultaneously and/or sequentially with the bone allograft. The additional agent or compound may be administered directly (e.g., by injection) to the site of the bone graft (e.g., before, after, and/or same time as the bone graft).

In a particular embodiment, the additional agent or compound is a therapeutic to aid in successful application of the bone allograft. The additional agent or compound may be, for example, a drug, a nucleic acid molecule, DNA, RNA, a polypeptide, a protein, a small molecule, biologic, growth factor, cytokine, chemokine, immunomodulating compound, signaling compound, antibodies, antibody fragments, and/or combinations thereof. In a particular embodiment, the additional agent or compound is a hydrophobic (e.g., with a hydrophobic polymer). Examples of therapeutic agents include but are not limited to agents that stimulate tissue growth and repair (e.g., agents that stimulate bone growth), anti-fibrotic agents, anti-inflammatory agents, pro-angiogenesis agents, and anti-microbial agents (including antibacterial, antiviral, and antifungal compounds).

In a particular embodiment, the additional agent or compound is an agent that stimulates bone growth. For example, the additional agent or compound may be, without limitation, a bone morphogenetic protein (e.g., BMP-2, BMP-6, BMP-7, BMP-12, BMP-9; particularly human; particularly BMP-2 and/or BMP-7 fragments, peptides, and/or analogs thereof). In a particular embodiment, the agent is BMP-2 or a BMP-2 peptide such as KIPKASSVPTELSAISTLYL (SEQ ID NO: 1). In a particular embodiment, the agent is a BMP-2 fragment (e.g., up to about 25, about 30, about 35, about 40, about 45, about 50 amino acids (e.g., consecutive), or more of BMP-2) comprising the knuckle epitope (e.g., amino acids 73-92 of BMP-2 or SEQ ID NO: 1). In a particular embodiment, the BMP-2 peptide is linked to a peptide of acidic amino acids (e.g., Asp and/or Glu; particularly about 3-10 or 5-10 amino acids such as E7, E8, D7, D8) and/or bisphosphonate (e.g., at the N-terminus).

In a particular embodiment, the additional agent or compound is an antifibrotic agent. In a particular embodiment, the antifibrotic agent is an inhibitor of TGF-β (e.g., TGF-01) signaling (e.g., a direct inhibitor of TGF-β (e.g., TGF-01)). Examples of antifibrotic agents include, without limitation: SB431542 (4-(5-benzol[1,3]dioxol-5-yl-4-pyrldin-2-yl-1H-imidazol-2-yl)-benzamide), SB505124 (2-[4-(1,3-benzodioxol-5-yl)-2-(1,1-dimethylethyl)-1H-imidazol-5-yl]-6-methyl-pyridine or 2-(5-benzo[1,3]dioxol-5-yl-2-tert-butyl-3H-imidazol-4-yl)-6-methylpyridine), paclitaxel (Hellal, et al. (2011) Science 331:928-931; Chen, et al. (2015) Drug Design Dev. Ther., 9:4869-4871; Zhou, et al. (2010) World J. Gastroenterol., 16:3330-3334), sirolimus, tumor necrosis factor related apoptosis-inducing ligand (TRAIL), corilagin, perifenidone, nintedanib, mitomycin C, 5-fluorouracil, simvastatin, and suramin. In a particular embodiment, the antifibrotic agents is selected from the group consisting of SB431542 (4-(5-benzol[1,3]dioxol-5-yl-4-pyrldin-2-yl-1H-imidazol-2-yl)-benzamide), paclitaxel, sirolimus, tumor necrosis factor related apoptosis-inducing ligand (TRAIL), corilagin, and perifenidone.

In a particular embodiment, the additional agent or compound is an antimicrobial (e.g., antibacterials, antivirals and/or antifungals). For example, the polymeric solution and/or coating may comprise an antibacterial or antibiotic.

In a particular embodiment, the additional agent or compound is an anti-inflammatory agent.

In a particular embodiment, the additional agent or compound is a pro-angiogenesis agent. Examples of pro-angiogenesis agents include, without limitation: vascular-endothelial growth factor (VEGF), basic fibroblast growth factor (bFGF), placental growth factor (PlGF), platelet-derived growth factor (PDGF), insulin-like growth factor (IGF) (e.g., insulin-like growth factor-1 (IGF-1)), VEGF peptides or mimicking peptides [e.g., DeRosa, et al. (2018) Arch. Biochem. Biophys., 660:72-86, incorporated by reference herein; e.g., QK peptides (e.g., encompassing/comprising amino acids 17-25 of the VEGF-A protein; e.g., KLTWQELQLKYKGI (SEQ ID NO: 2), KLTWQELQLKYKGIGGG (SEQ ID NO: 3), or KLTWQELQLKYKGIGGGEEEEEEE (SEQ ID NO: 4)); e.g., PR1P (prominin-1-derived peptide) peptides (e.g., Adini et al. (2017) Angiogenesis 20(3):399-408, incorporated by reference herein, e.g., DRVQRQTTTVVA (SEQ ID NO: 5); e.g., Lv peptide (e.g., Shi et al. (2019) J. Amer. Heart Assoc., 8:22; incorporated by reference herein; e.g., Gene ID: 196740 (e.g., a.a. 55-94); e.g., DSLLAVRWFFAHSFDSQEALMVKMTKLRVVQYYGNFSRSA (SEQ ID NO: 6); e.g., RoY peptides (e.g., Shu et al. (2015) ACS Appl. Mater. Interfaces, 7:12, incorporated by reference herein; e.g., YPHIDSLGHWRR (SEQ ID NO: 7)], periostin peptides (Kim, et al. (2017) PLoS ONE 12(11):e0187464; incorporated by reference herein; e.g., comprising amino acids 142-151 (WDNLDSDIRR (SEQ ID NO: 8)) or 136-151 (APSNEAWDNLDSDIRR (SEQ ID NO: 9)) of periostin) and fragments or derivatives thereof (see, e.g., Risau, W. (1990) Prog. Growth Factor Res., 2(1):71-79; incorporated herein by reference). In a particular embodiment, the pro-angiogenesis agent is an angiogenic peptide. Examples of angiogenic peptides include, without limitation, vascular endothelial growth factor (VEGF) peptides or mimicking peptides and periostin peptides.

In certain embodiments of the instant invention, the polymeric solution and/or coating comprises at least one agent that stimulates bone growth (e.g., BMP-2 peptide) and at least one antifibrotic agent (e.g., corilagin). The agent that stimulates bone growth and the antifibrotic agent maybe contained within the same layer or coating and/or may be contained within separate layers or coatings. For example, the agent that stimulates bone growth (e.g., BMP-2 peptide) may be contained within an outer layer and the antifibrotic agent (e.g., corilagin) may be contained within an inner layer.

The bone allografts, particularly after synthesis, may be washed or rinsed in water and/or a desired carrier or buffer (e.g., a pharmaceutically or biologically acceptable carrier). The bone allografts may also be stored in a cold solution, lyophilized and/or freeze-dried. In a particular embodiment, the bone allografts are freeze-dried after synthesis (e.g., to remove any solvent). The bone allografts of the instant invention may also be sterilized. For example, the bone allografts can be sterilized using various methods (e.g., by treating with ethylene oxide gas, gamma irradiation, or 70% ethanol).

The bone allografts of the instant invention may also comprise cells and/or be administered with cells. In a particular embodiment, the cells are autologous to the subject to be treated with the bone allograft. The bone allografts may be coated with or comprise any cell type. In a particular embodiment, the cells comprise stem cells or mesenchymal stem cells. In a particular embodiment, the cells comprise bone marrow-derived mesenchymal stem/stromal cells (BMSC). In a particular embodiment, the cells comprise dermal fibroblasts. In a particular embodiment, the bone allograft comprises a tissue sample (e.g., minced tissue), such as a bone sample. The cells or tissue may be cultured with the bone allograft (e.g., the cells or tissue may be cultured for sufficient time to allow for growth on and/or infiltration onto the bone allograft). For example, the cells or tissue may be cultured with the coated bone graft for 1 day, 2 days, 3 days, 4 days, 5 days, or more.

In accordance with another aspect of the instant invention, methods of synthesizing the bone grafts (e.g., allografts) described herein are provided. In a particular embodiment, the method comprises electrospraying one or more solutions comprising a polymer and, optionally, one or more additional agents or compounds, onto a bone allograft, thereby synthesizing the coated bone allograft. In a particular embodiment, the method further comprises freeze drying and/or lyophilizing the coated bone allograft. In a particular embodiment, the method further comprises modifying the coated bone allograft as described herein (e.g., cellular coating, mineralization, etc.). In a particular embodiment, the method further comprises washing and/or sterilizing the coated bone allograft. In a particular embodiment, the method further comprises obtaining the bone graft (e.g., from a subject or the patient (e.g., for an autograft)) prior to electrospraying. In a particular embodiment, the method further comprises inserting the coated bone graft into a subject to be treated, optionally further comprising administering at least one additional agent or compound as described herein.

In accordance with another aspect of the instant invention, methods of treating a subject with a bone defect are provided. The methods comprise administering (e.g., transplanting) a coated bone graft (e.g., allograft) of the instant invention to the subject. In a particular embodiment, the coated bone graft is inserted to the site of the bone defect. In a particular embodiment, the method comprises synthesizing the bone allograft prior to administration or transplantation. In a particular embodiment, the method further comprises administering at least one additional agent or compound as described herein. When administered separately, the bone graft may be administered simultaneously and/or sequentially with the additional agent or compound. The methods may comprise the administration of one or more bone grafts. When more than one bone graft is administered, the bone grafts may be administered simultaneously and/or sequentially.

The bone grafts of the present invention may be administered by any method—typically by surgical means. The term “patient” as used herein refers to human or animal subjects.

The compositions of the instant invention may be conveniently formulated for administration with any pharmaceutically acceptable carrier(s). For example, the agents may be formulated with an acceptable medium such as water, buffered saline, ethanol, polyol (for example, glycerol, propylene glycol, liquid polyethylene glycol and the like), dimethyl sulfoxide (DMSO), oils, detergents, suspending agents or suitable mixtures thereof. The concentration of the agent or compound in the chosen medium may be varied and the medium may be chosen based on the desired route of administration of the pharmaceutical preparation. Except insofar as any conventional media or agent is incompatible with the agents to be administered, its use in the pharmaceutical preparation is contemplated.

Compositions of the instant invention may be administered by any method. For example, the compositions of the instant invention can be administered, without limitation, parenterally, subcutaneously, orally, topically (ex. using a cream or spray), pulmonarily, rectally, vaginally, intravenously, intraperitoneally, intrathecally, intracerbrally, epidurally, intramuscularly, intradermally, intratumoral, intracarotidly, or by direct injection (e.g., a localized injection into a specific tissue or organ (e.g., to the site of the bone graft)). Selection of a suitable pharmaceutical preparation will also depend upon the mode of administration chosen. For example, the compositions of the invention may be administered parenterally. In this instance, a pharmaceutical preparation comprises the agent or compound dispersed in a medium that is compatible with the parenteral injection. The agent or compound may be formulated in a variety of solutions and formats, such as, without limitation, a cream or ointment, a spray such as an aerosol, a powder, colloidal dispersion, emulsion, gels, and a liquid for injection or other form of administration.

Pharmaceutical compositions containing an agent of the present invention as the active ingredient in intimate admixture with a pharmaceutically acceptable carrier can be prepared according to conventional pharmaceutical compounding techniques. The carrier may take a wide variety of forms depending on the form of preparation desired for administration, e.g., parenterally.

### Definitions

The singular forms “a,” “an,” and “the” include plural referents unless the context clearly dictates otherwise.

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, preservative (e.g., Thimersol, benzyl alcohol), anti-oxidant (e.g., ascorbic acid, sodium metabisulfite), solubilizer (e.g., polysorbate 80), emulsifier, buffer (e.g., TrisHCl, acetate, phosphate), water, aqueous solutions, oils, bulking substance (e.g., lactose, mannitol), excipient, auxiliary agent or vehicle with which an active agent of the present invention is administered. Suitable pharmaceutical carriers are described in “Remington's Pharmaceutical Sciences” by E. W. Martin (Mack Publishing Co., Easton, Pa.); Gennaro, A. R., Remington: The Science and Practice of Pharmacy, (Lippincott, Williams and Wilkins); Liberman, et al., Eds., Pharmaceutical Dosage Forms, Marcel Decker, New York, N.Y.; and Kibbe, et al., Eds., Handbook of Pharmaceutical Excipients (3rd Ed.), American Pharmaceutical Association, Washington.

As used herein, the term “polymer” denotes molecules formed from the chemical union of two or more repeating units or monomers. The term “block copolymer” most simply refers to conjugates of at least two different polymer segments, wherein each polymer segment comprises two or more adjacent units of the same kind.

“Hydrophobic” designates a preference for apolar environments (e.g., a hydrophobic substance or moiety is more readily dissolved in or wetted by non-polar solvents, such as hydrocarbons, than by water). In a particular embodiment, hydrophobic polymers may have aqueous solubility less than about 1% wt. at 37° C. In a particular embodiment, polymers that at 1% solution in bi-distilled water have a cloud point below about 37° C., particularly below about 34° C., may be considered hydrophobic.

As used herein, the term “hydrophilic” means the ability to dissolve in water. In a particular embodiment, polymers that at 1% solution in bi-distilled water have a cloud point above about 37° C., particularly above about 40° C., may be considered hydrophilic.

As used herein, the term “amphiphilic” means the ability to dissolve in both water and lipids/apolar environments. Typically, an amphiphilic compound comprises a hydrophilic portion and a hydrophobic portion.

The term “antimicrobials” as used herein indicates a substance that kills or inhibits the growth of microorganisms such as bacteria, fungi, viruses, or protozoans.

As used herein, the term “antiviral” refers to a substance that destroys a virus and/or suppresses replication (reproduction) of the virus. For example, an antiviral may inhibit and or prevent production of viral particles, maturation of viral particles, viral attachment, viral uptake into cells, viral assembly, viral release/budding, viral integration, etc.

As used herein, the term “antibiotic” refers to antibacterial agents for use in mammalian, particularly human, therapy. Antibiotics include, without limitation, beta-lactams (e.g., penicillin, ampicillin, oxacillin, cloxacillin, methicillin, and cephalosporin), carbacephems, cephamycins, carbapenems, monobactams, aminoglycosides (e.g., gentamycin, tobramycin), glycopeptides (e.g., vancomycin), quinolones (e.g., ciprofloxacin), moenomycin, tetracyclines, macrolides (e.g., erythromycin), fluoroquinolones, oxazolidinones (e.g., linezolid), lipopetides (e.g., daptomycin), aminocoumarin (e.g., novobiocin), co-trimoxazole (e.g., trimethoprim and sulfamethoxazole), lincosamides (e.g., clindamycin and lincomycin), polypeptides (e.g., colistin), and derivatives thereof.

As used herein, an “anti-inflammatory agent” refers to compounds for the treatment or inhibition of inflammation. Anti-inflammatory agents include, without limitation, non-steroidal anti-inflammatory drugs (NSAIDs; e.g., aspirin, ibuprofen, naproxen, methyl salicylate, diflunisal, indomethacin, sulindac, diclofenac, ketoprofen, ketorolac, carprofen, fenoprofen, mefenamic acid, piroxicam, meloxicam, methotrexate, celecoxib, valdecoxib, parecoxib, etoricoxib, and nimesulide), corticosteroids (e.g., prednisone, betamethasone, budesonide, cortisone, dexamethasone, hydrocortisone, methylprednisolone, prednisolone, tramcinolone, and fluticasone), rapamycin, acetaminophen, glucocorticoids, steroids, beta-agonists, anticholinergic agents, methyl xanthines, gold injections (e.g., sodium aurothiomalate), sulphasalazine, and dapsone.

As used herein, the term “subject” refers to an animal, particularly a mammal, particularly a human.

As used herein, the term “prevent” refers to the prophylactic treatment of a subject who is at risk of developing a condition resulting in a decrease in the probability that the subject will develop the condition.

The term “treat” as used herein refers to any type of treatment that imparts a benefit to a patient afflicted with a disease, including improvement in the condition of the patient (e.g., in one or more symptoms), delay in the progression of the condition, etc.

As used herein, the term “analgesic” refers to an agent that lessens, alleviates, reduces, relieves, or extinguishes pain in an area of a subject's body (i.e., an analgesic has the ability to reduce or eliminate pain and/or the perception of pain).

As used herein, the term “small molecule” refers to a substance or compound that has a relatively low molecular weight (e.g., less than 2,000). Typically, small molecules are organic, but are not proteins, polypeptides, or nucleic acids.

As used herein, the term “allograft” refers to a tissue graft from a donor of the same species as the recipient. As used herein, the term “autograft” refers to a tissue graft from the same individual.

The following examples illustrate certain embodiments of the invention. They are not intended to limit the invention in any way.

### Example 1

### Materials and Methods

### Materials

The following chemicals were used: poly(lactide-co-glycolide) (PLGA) 50:50, Mw 30000-60000 (Lactel® absorbable polymers; Evonik, Birmingham, Ala.), dichloromethane (DCM, Acros Organics, Fair Lawn, N.J.), polycaprolactone (PCL) [Mw=80 kDa] (Sigma Aldrich, St. Louis, Mo.), gelatin (Sigma Aldrich), and hexafluoro-2-propanol (HFIP) (Oakwood Chemical, Estill, S.C.).

### Experimental Animals

All in vivo experiments were performed using adult 8-12 week old C57BL/6 mice housed in pathogen-free, temperature and humidity controlled facilities with a 12 hour day-night cycle in the vivarium at the University of Rochester Medical Center. All cages contained wood shavings, bedding and a cardboard tube for environmental enrichment. All experimental procedures were reviewed and approved by the University Committee on Animal Resources. General anesthesia, and analgesia procedures were performed based on the mouse formulary provided by the University Committee on Animal Resources. The animals' health status was monitored throughout the experiments by experienced veterinarians according to the Guide for the Care and Use of Laboratory Animals outlined by the National Institute of Health.

### Preparation of Bone Allograft Coatings Via Polymer-Mediated Electrospray Deposition

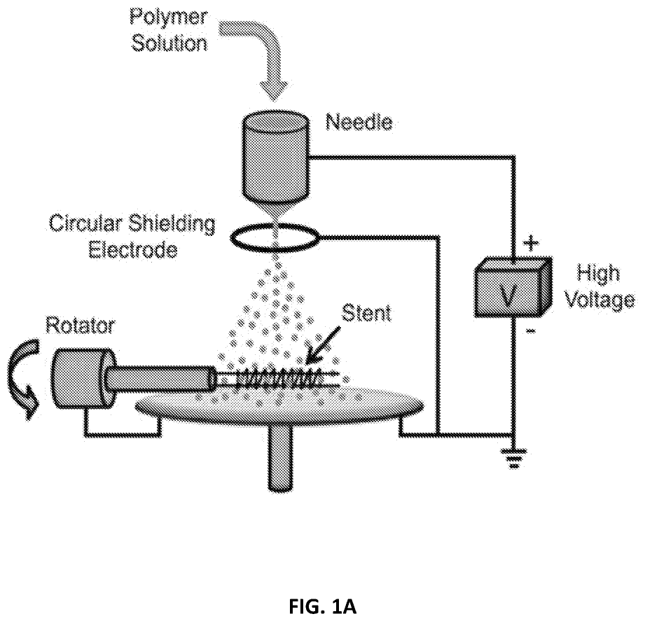

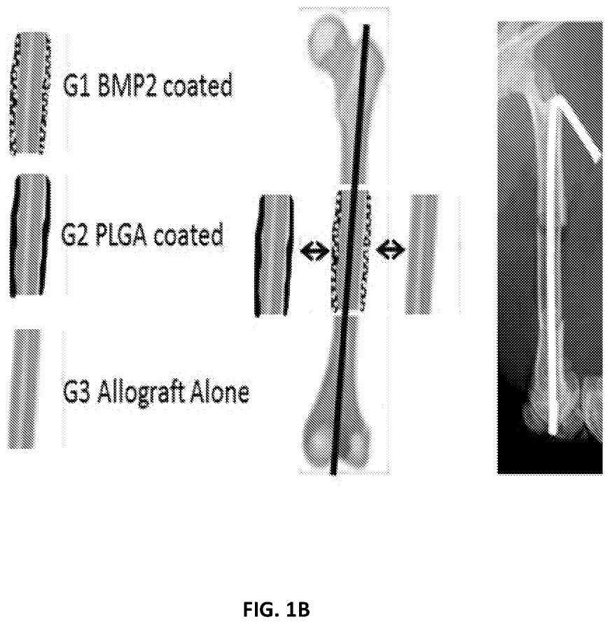

A schematic illustrating the setup of electrospray deposition is shown (FIG. 1A). A voltage of 6.0-8.0 kV was applied to the nozzle and a voltage of 4.0 kV was applied to the metal ring to stabilize the electrospray towards the bone allograft on top of the rotating motor (EURO-ST D, Kika Labortechnik, Staufen, Germany). This was utilized as the high viscosity of the polymeric solution can cause partial clogging of the capillary, thus resulting in deviance in spraying direction. The distance between the spinneret and the rotating bone sample bone allograft graft was about 3 cm, and distance between the spinneret and grounded Al foil collector was of 15 cm. The syringe pump (Stoelting Co., Chicago, Ill.) was set at 1 mL/hour for all samples. A stable cone-jet was formed during electrospraying. Following electrospray deposition, the coated bone samples were left overnight in the hood and then freeze-dried to ensure complete solvent removal.

To stimulate osteogenic differentiation and bone formation, a short-chain BMP-2 mimetic peptide (KIPKASSVPTELSAISTLYL (SEQ ID NO: 1), Genscript, Piscataway, N.J.) was incorporated into the coatings. The BMP-2 mimetic peptide binds and activates the receptors of BMPs (Kanie, et al. (2016) Materials (Basel) 9(9):730; Saito, et al. (2003) Biochim. Biophys. Acta 1651:60-7; Senta, et al. (2011) Can. J. Chem. Eng., 89:227-39; Madl, et al. (2014) Biomacromolecules 15:445-55). The peptide (5 mg) was dispersed in 5% (w/v) of poly(lactide-co-glycolide) (PLGA) (Lactel® absorbable polymers 50:50, Mw 30000-60000) in dichloromethane (DCM) in glass vials. PLGA in DCM with or without containing peptide was used for electrospray coating of the allografts. The polymer to peptide weight ratio was 50:1. The bone allografts were weighed initially (W₀) and weighed after coating (W₁), to maintain the uniform coating (W₁-W₀≈3 mg) for both in vitro and in vivo studies.

### Characterization of Bone Allograft Coatings and Peptide Release

The coated bone allograft was characterized by scanning electron microscopy (SEM) to measure the thickness. Profilm3D® technology conducted by Instruments Group/Filmetrics, KLA Corporation was used to examine the polymer coating on the bone substrates. The 3D optical profiler consisted of various objectives from 5× to 100×. Here, 20× (Nikon CF IC Epi Plan) with working distance of 4.7 mm was used to measure the coating thickness. The resulting graph was stitched using software provided by Profilm3D® (Filmetrics; San Diego, Calif.).

For quantification of peptide loading and release kinetics, 5 mg FITC labeled BMP-2 peptide was dispersed in 5% (w/v) of PLGA in DCM in glass vials and used for electrospray-mediated allograft coating. The amount of BMP-2 peptide loaded per graft was calculated from the difference in the weights of the bone samples before and after electrospray coating as well as accounting for the polymer:peptide ratio of 50:1 (Boda, et al. (2019) Acta Biomater., 85:282-93). The duration of the electrospray coating was adjusted to achieve an increase of 2-3 mg in the weights of the electrospray coated bone samples as compared to the corresponding uncoated ones. The FITC-labeled peptide release from the bone allograft was recorded by measuring the fluorescence intensities of the buffer aliquots at regular intervals using excitation and emission filters of 485 and 528 nm, respectively.

Similarly, to measure the peptide loading and release kinetics from PCL-gelatin coated allografts, the PCL-gelatin HFIP solution containing 5 mg FITC labeled BMP-2 peptide was used for coating. PCL-Gelatin (2.5 wt % each) and 5 mg of BMP-2 peptide were mixed by ultrasonication. The polymer to peptide weight ratio was 50:1. The above solution was electrospray deposited on the bone allografts and freeze-dried as described. In the case of PCL-Gelatin coatings, the samples were cross-linked with glutaraldehyde vapors from a 25 wt % ethanolic solution overnight for ˜24 hours. The peptide release from the PCL-gelatin bone allograft was recorded by measuring the fluorescence intensities of the buffer aliquots at regular intervals (Boda, et al. (2019) Acta Biomater., 85:282-93).

### Surgical Procedures for Segmental Femoral Bone Allograft Model

A 4 mm segmental femoral bone graft transplantation model was used to evaluate the efficacy of coated allografts for reconstruction of long bone defect repair (FIG. 1B) (Huang, et al. (2014) Mol. Ther., 22:430-9; Zhang, et al. (2005) J. Bone Miner. Res., 20:2124-37; Xie, et al. (2007) Tissue Eng., 13:435-45). Briefly, mice were anesthetized via intraperitoneal injection with a combination of ketamine and xylazine. A transverse unilateral osteotomy was performed to remove a 4 mm femoral diaphyseal shaft using a bone saw. A devitalized allograft or a devitalized allograft wrapped with nanofiber sheets with or without bone marrow-derived mesenchymal stem/stromal cells (BMSC)—seeding was used to repair the defect. The grafts were secured by a 22-gauge metal pin placed through the intramedullary marrow cavity. Allografts were prepared from an outbred strain of FVB and washed extensively with phosphate buffered saline to remove periosteum, bone marrow cells and cell debris. The grafts were further washed with 70% ethanol, soaked in PBS containing a cocktail of penicillin and streptomycin and frozen at −80° for at least 1 week. During the postoperative period, pain was relieved by a subcutaneous administration of butamorphine (Pfizer Animal Health; 5 mg/kg twice daily for 2 days).

### Experimental Animal Groups

The murine allograft model demonstrates poor graft integration and biomechanics up to week 9 post-surgery (Hoffman, et al. (2013) Biomaterials 34:8887-98; Xie, et al. (2007) Tissue Eng., 13:435-45). Accordingly, week 5 post-surgery was chosen to conduct MicroCT analyses and week 7 was chosen for biomechanical testing. A total of 24 mice in 4 groups (allograft, allograft with PLGA coating, and allograft with BMP-2 peptide loaded PLGA, n=6) were included in the MicroCT analyses. These same samples were used for analysis of allograft surface mineralization using Amira software as well as histologic and histomorphometric analyses to determine graft healing. Additional groups of samples were used for torsional biomechanical testing at week 7 to determine the functional integration of the allograft with host bone. At least five mice per group were used for evaluation.

### Evaluation of Femoral Allograft Healing by MicroCT

Samples were scanned by ScancoVivaCT 40 system (Scanco Medical AG, Bassersdorf, Switzerland) at 12.5-micron isotropic resolution. Images were reconstructed to allow 3-dimensional structural rendering of the calluses at a standardized threshold corresponding to 750 mgHA/cm³ based on a phantom of known HA concentrations. To evaluate bone formation, contour lines were drawn in the 2-dimensional slice images to exclude the allograft and the old host cortical bone. New bone volume on the side of the host and donor bone, as well as in the total callus in grafted samples was calculated, respectively (Zhang, et al. (2005) J. Bone Miner. Res., 20:2124-37; Xie, et al. (2007) Tissue Eng., 13:435-45).

### Analysis of New Bone Deposition on Allograft Surface

Measurement of allograft surface deposited with newly formed bone was performed based on high resolution MicroCT scans using Amira software and custom algorithms. Briefly, an Amira build-in algorithm was used to isolate new bone within 50 μm distance from allograft surface. All newly formed bone on or immediate adjacent to the allograft surface were segmented via thresholding to include newly formed bone (lower density) but exclude allograft (higher density). A new bone shell covering the allograft surface in three experimental groups was generated and used to calculate the inner surface area that overlaying the outer surface of the allograft. The percent allograft surface overlaid by newly deposited bone was then calculated by counting pixels of the inner surface of the new bone shell and the outer surface of the allograft using marching cubes algorithm and triangle mesh calculation of the surface area (Lorensen, et al. (1987) Computer Graphics (SIGGRAPH 87 Proceedings) 21:163-70; Lewiner, et al. (2003) J. Graphics Tools 8:1-15). The ratio of the two surface areas reflects fraction of mineralized surface of the bone allograft.

### Evaluation of Femoral Allograft Healing Via Histology and Histomorphometric Analyses

At the end point of the experiment, mice were perfused with 4% paraformaldehyde followed by an additional tissue fixation for 2 days. The specimens were decalcified in 10% EDTA and processed for frozen sectioning. Mid-sagittal frozen sections (20 microns thick) or paraffin-embedded tissue sections (6 microns thick) were stained with hematoxylin & eosin plus alcian blue hematoxylin/orange G, or tartrate-resistant acidic phosphatase (TRAP) (Huang, et al. (2014) Mol. Ther., 22:430-9; Zhang, et al. (2005) J. Bone Miner. Res., 20:2124-37; Xie, et al. (2007) Tissue Eng., 13:435-45). Tissue sections were digitalized via Olympus VS110™ Virtual Slide Scanning System (Olympus, Tokyo, Japan). Histomorphometric analyses of bone, cartilage, and fibrotic tissue formation were performed in the VisioPharm® Image Analysis Software via color-based semi-manual segmentation of different tissue component of the healing callus (Horsholm, Denmark) (Dhillon, et al. (2014) Methods Mol. Biol., 1130:45-59; Zhang, et al. (2016) Bone Res., 4:15037). Percentage area of bone, cartilage, bone marrow and fibrotic tissue within the area of callus were calculated to illustrate the difference among each group of samples.

### Evaluation of Femoral Allograft Healing Via Torsional Biomechanical Testing

Following sacrifice, the tibia was isolated and cleaned of excess soft tissue. Tibias were stored at 4° C. in phosphate saline buffer overnight, prior to torsional biomechanical testing. The ends of the tibias were cemented (Bosworth Company) in aluminum tube holders and tested using an EnduraTec TestBench™ system (Bose Corporation, Eden Prairie, Minn.). The tibias were tested at a rate of ideg/second, in torsion, until failure. The rotational data was converted to radians/mm to complete the torsional rigidity analysis. The ultimate torque and the torsional rigidity were determined based on the load-to-failure curve generated. The intact femurs from non-surgical mice were used as controls for allografted bone in torsional biomechanical testing.

### Immunofluorescent and Immunohistochemical Analysis

For SMAD3, SMAD1/5 immunofluorescense staining, the slides were deparaffinzed, rehydrated into water. Next, the samples were treated with 3% bovine albumin in PBS and then stained with p-Smad3 (1:100 dilution, Rockland, Pa.) and p-Smad5 antibody (1:100 dilution, Cell Signaling, MA) overnight at 4° C. The samples were incubated with secondary antibody (Alexa Fluor 546 dye, Thermofisher) for two hours at room temperature after 3 times of wash. The samples were imaged via Olympus VS110™ Virtual Slide Scanning System (Olympus, Tokyo, Japan).

### Statistical Analysis

All data are shown as the mean±standard deviation. Statistical analysis was analyzed by one-way ANOVA in GraphPad Prism (GraphPad Prism, San Diego, Calif.). A p value <0.05 was considered statistically significant.

### Results

### Characterization of the BMP-2 Peptide-Loaded Thin Polymer Coating on Bone Allografts

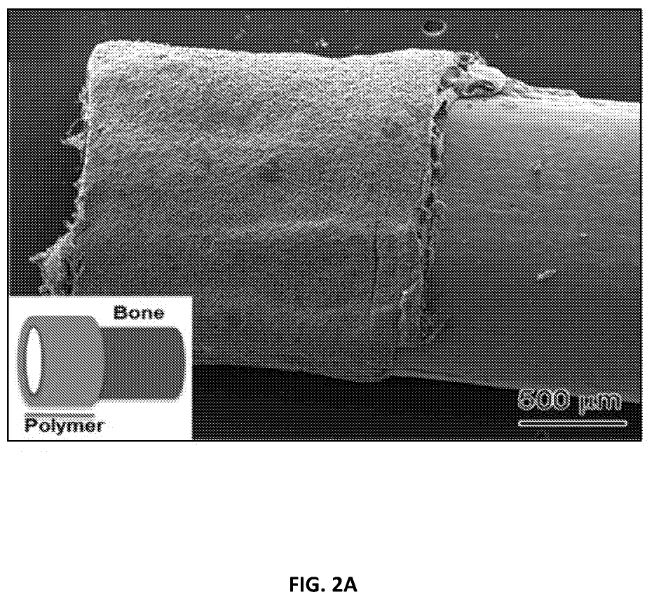

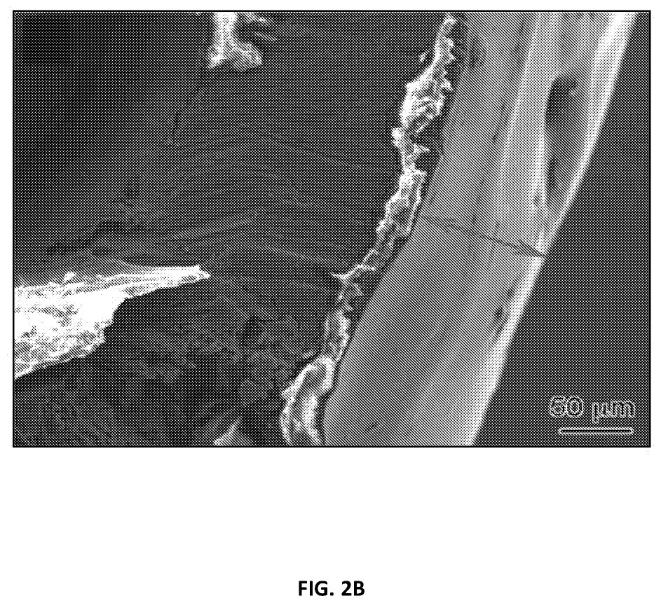

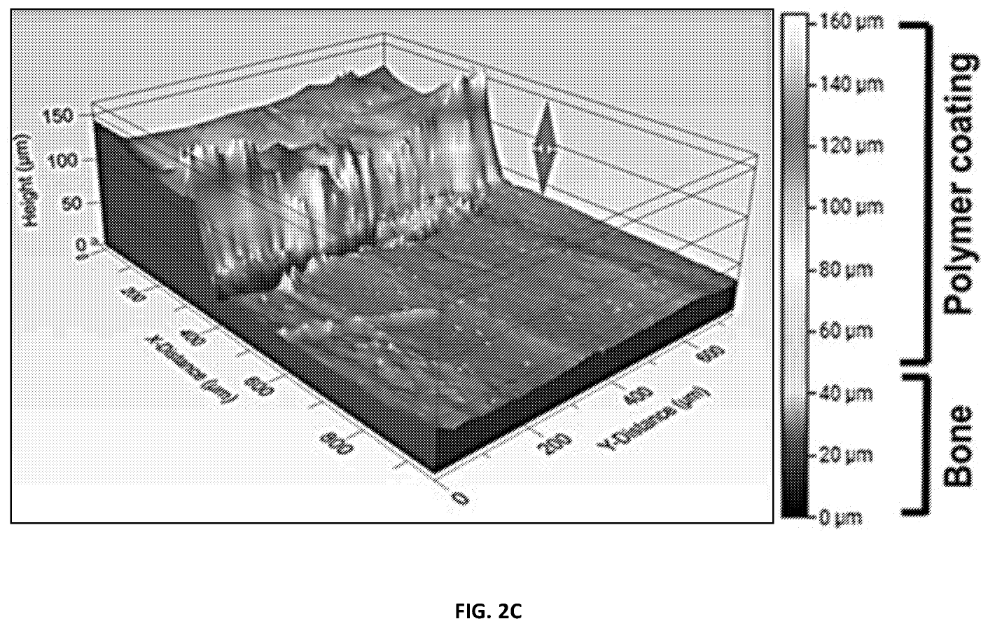

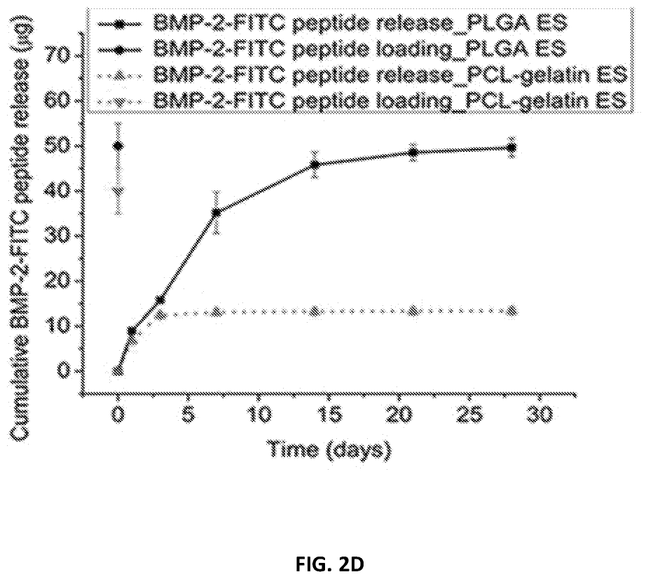

Allograft surface coating was characterized by SEM. PLGA coated bone allografts was collected with a portion of the graft covered with tape while electrospraying. Subsequent removal of the tape revealed the details of electrospray-coated bone surface via SEM. As shown, uniform BMP-2 peptide-loaded PLGA coating was formed on the surface of bone allografts (FIG. 2A). The cross-sectional SEM image further illustrated the coating of approximately ≈100 μm in thickness (FIG. 2B). To further examine the uniformity of the coating, the optical profilometry was performed. The Profilm3D® filmetrics system uses state-of-the-art white light interferometry (WLI) and is capable of measuring the surface profiles and roughness down to 0.05 μm. As shown via Profilm3D® images (FIG. 2C), PLGA coated bone allografts had a uniform distribution of polymer coatings on bone allografts. The coating was approximately 100 μm thick, which was consistent with SEM results.

To determine the capability of polymer-dependent controlled release of the peptide from the allograft surface coating, FITC labeled BMP-2 peptide was loaded in PLGA and PCL-gelatin, the two coating polymers with vastly different degradation profile (Makadia, et al. (2011) Polymers 3:1377-97; Woodruff, et al. (2010) Prog. Polym. Sci., 35:1217-56). The peptide loading and release were quantified following incubation of the allografts at 37° C. in Tris-buffered saline over a period of 30 days. As shown (FIG. 2D), the loading of FITC labeled peptide on allograft was similar in PLGA and PCL-gelatin media (ca. 50 μg). However, a sustained release profile of BMP-2 peptide was recorded with >90% of the peptide released over a period of 30 days from PLGA coated bone allografts. In comparison, only ˜30% BMP-2 peptide release was recorded from the PCL-Gelatin within the same time interval, indicating that the PCL-Gelatin peptide coatings exhibited a retarded peptide release perhaps due to the prolonged degradation of PCL as well as glutaraldehyde cross-linking of the coatings.

### BMP-2 Peptide-Loaded Bone Allografts Showed Improved Healing in Repair of a Segment Femoral Bone Defect Model in Mice

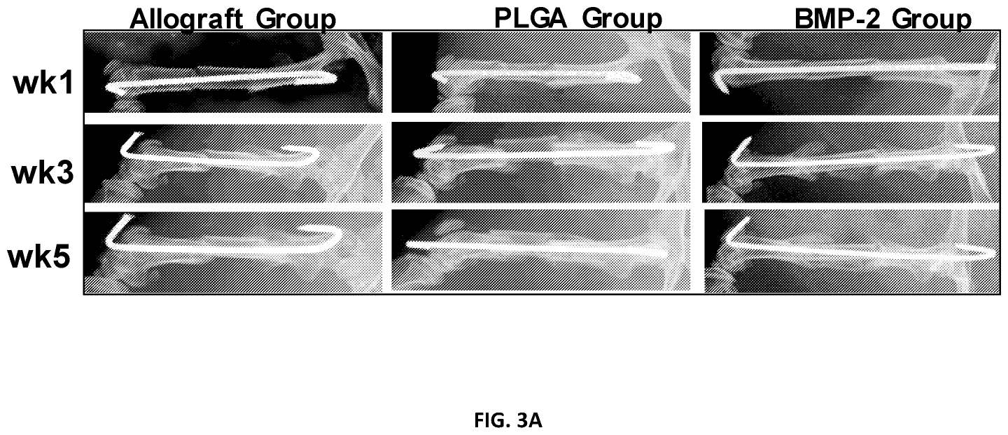

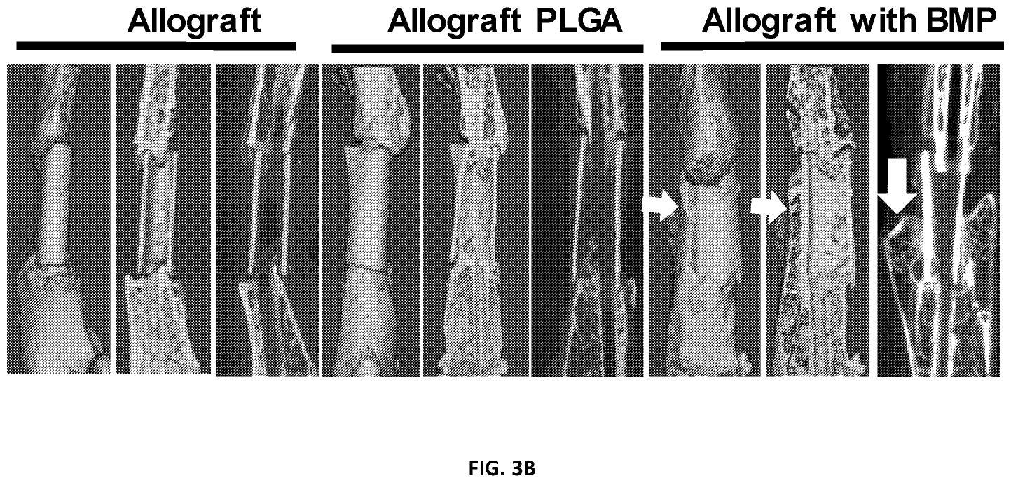

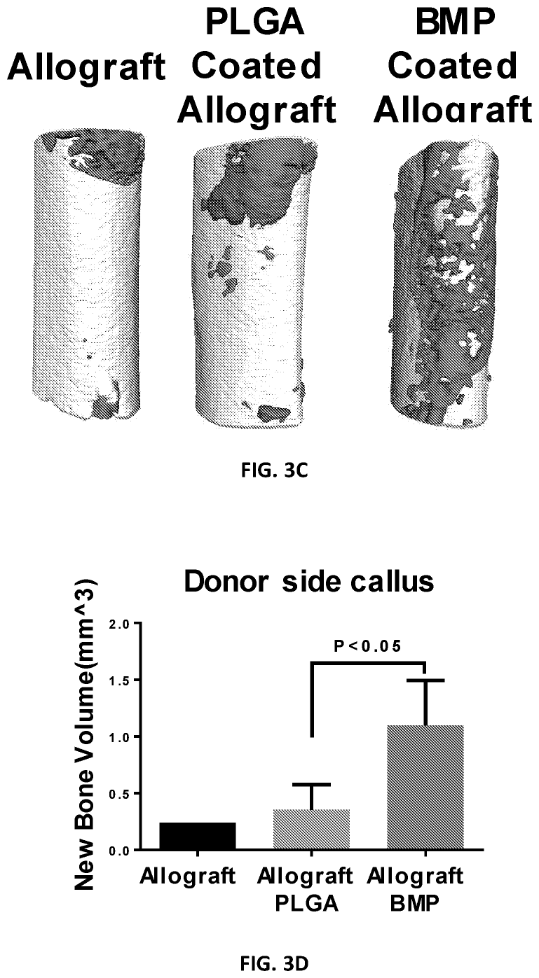

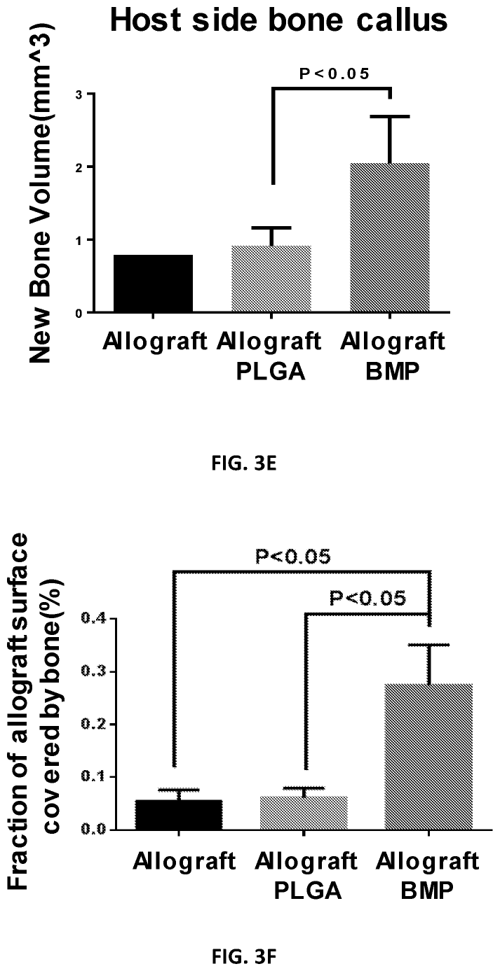

A murine segmental bone graft model has been established that recapitulates the most prominent features of bone graft healing in humans (Zhang, et al. (2005) J. Bone Miner. Res., 20:2124-37; Tiyapatanaputi, et al. (2004) J. Orthop. Res., 22:1254-60). This model allows for the study of the molecular and cellular events that govern allograft healing and remodeling (Xie, et al. (2008) Bone 43:1075-83; Xie, et al. (2008) J. Bone Joint Surg. Am., 90 Suppl 1:9-13; Wang, et al. (2010) Am. J. Pathol., 177:3100-11; Wang, et al. (2011) Bone 48:524-32). This model also allows for testing tissue-engineered bone allografts for repair of critical-sized bone defects (Koefoed, et al. (2005) Mol. Ther., 12:212-8; Hoffman, et al. (2013) Biomaterials 34:8887-98; Wang, et al. (2018) Biomaterials 182:279-88; Huang, et al. (2014) Mol. Ther., 22:430-9; Xie, et al. (2007) Tissue Eng., 13:435-45; Ito, et al. (2005) Nat. Med., 11:291-7; Zhang, et al. (2008) Clin. Orthop. Relat. Res., 466:1777-87). To test the effects of BMP-2 peptide-loaded allografts, healing was examined in a 4-mm segmental defect created in femurs of C57BL6 mice. Longitudinal X-ray examination showed significant more callus formation close to or on the allograft surface in samples treated with BMP peptide coated allografts (FIG. 3A, arrows). MicroCT reconstruction of the defect at week 5 post-surgery showed that compared with allografts coated with PLGA, BMP-2 peptide-coated allografts demonstrated significantly enhanced bone formation at the repair sites (FIG. 3B). Newly formed bone was found immediately adjacent to the allograft surface in many samples examined (FIG. 3B, arrows). Quantitative MicroCT analyses showed 3.1 and 2.3-fold increase of new bone at the donor and host side callus, respectively, at week 5 post-surgery (FIGS. 3D and 3E, n=6, p<0.05).

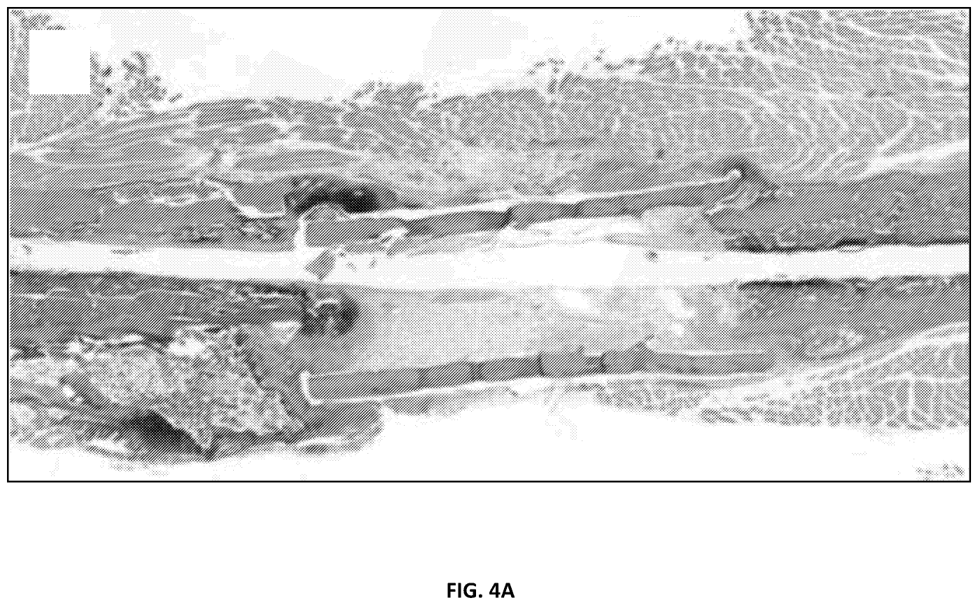

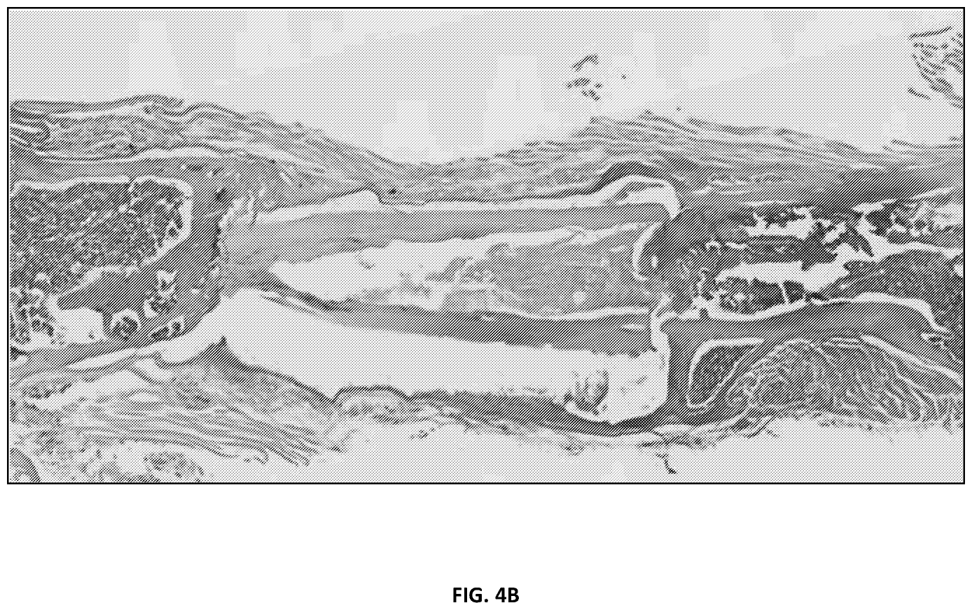

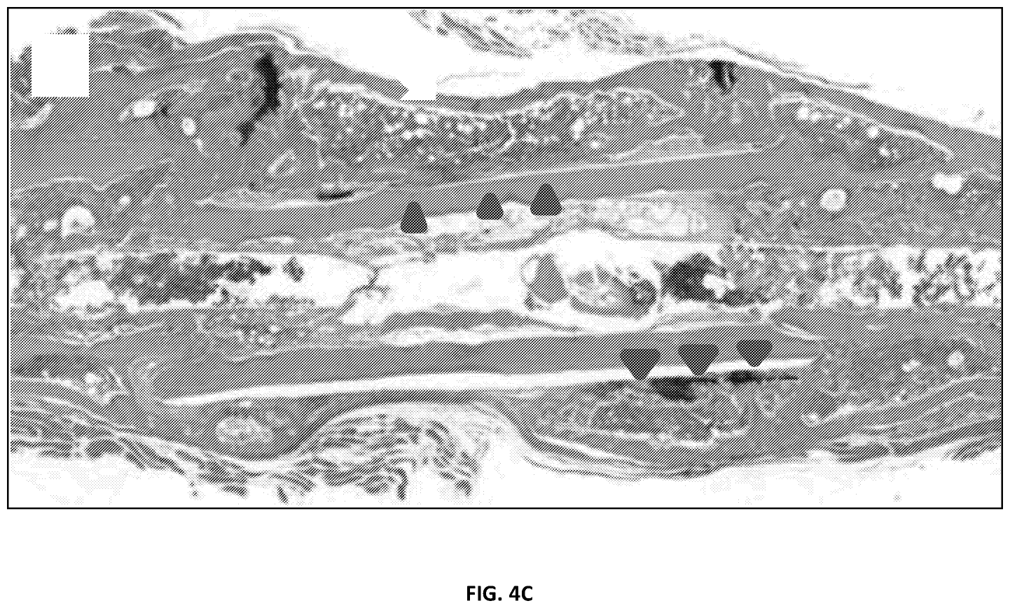

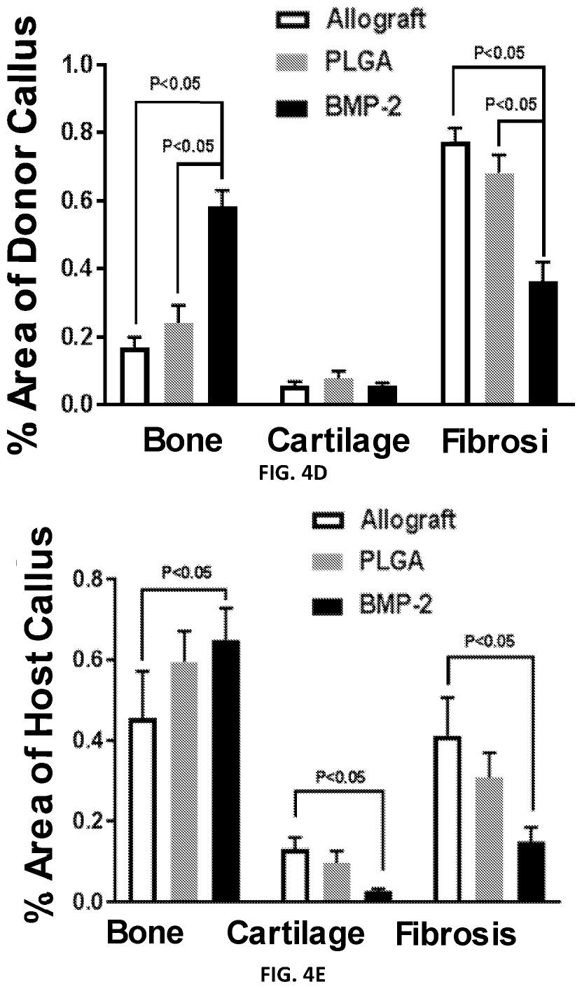

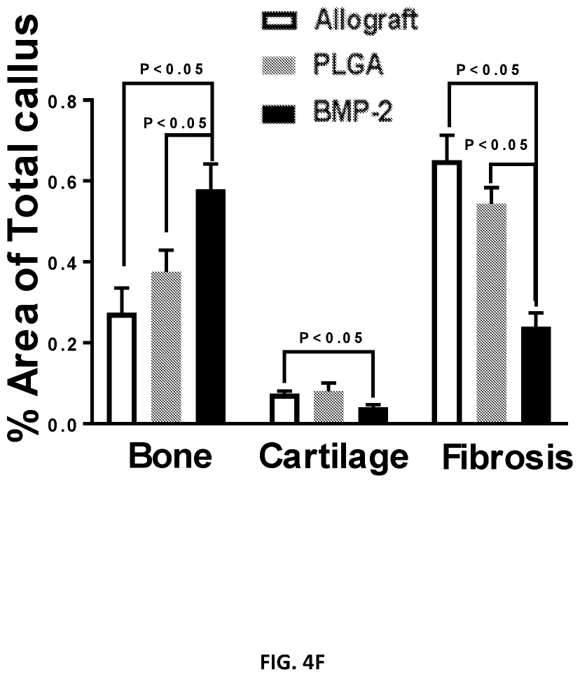

To determine the direct deposition of newly formed bone on the surface of bone allograft, which is an indication of allograft integration and capability of remodeling itself, the minimum graft surface area fraction that was covered by new bone shell was examined and calculated in three different groups using MicroCT 2D image stacks. As shown (FIG. 3C), allograft coated with BMP-2 peptide showed significantly increased new bone coverage over the allograft outer surface than allograft alone or allograft coated with PLGA only. Quantitative analyses showed about 5 fold increase of the allograft surface overlaid by new bone in BMP-2 peptide coated groups as compared with the other two groups (FIG. 3F, p<0.05, n=6). The direct induction of bone on allograft surface was seen in near all allograft samples that coated with BMP-2 peptide. Further histologic analyses were conducted at week 5 post-implantation. Significant fibrotic tissue and immature fibrocartilage on the surface of allografts and at the cortical bone junctions were observed in all three groups (FIG. 4A-4C). However, comparing to the control groups, BMP-2 peptide-coated grafts showed remarkable osteoinductive activity leading to enhanced bone callus formation directly on top of the bone allografts (FIG. 4C, arrows). Quantitative histomorphometric analyses showed 4.2- and 2.3-fold induction of bone formation at the donor and host site, respectively (FIG. 4D-4E, n=6, p<0.05). Total percent bone in area of callus was also significantly enhanced by 2 fold (FIG. 4F, p<0.05). With increased bone formation, the percentage area of fibrotic tissue was reduced in BMP-2 treated group, indicating the antagonism between osteogenesis and fibrosis.

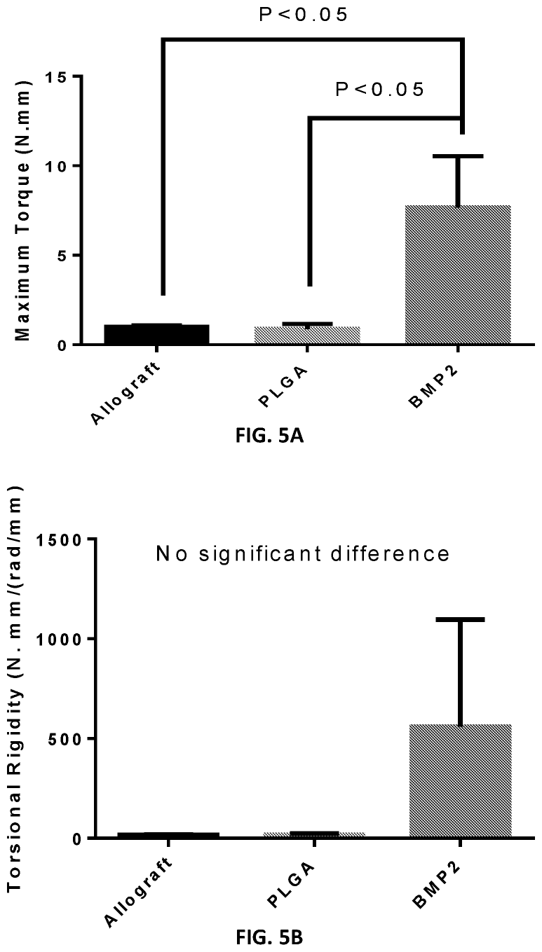

Lastly, torsional biomechanical testing was conducted at week 7 post surgery to determine the functional healing of the three groups of allografts. As shown, compared to allografts and allografts coated with PLGA, allografts coated with BMP-2 peptide via PLGA demonstrated significantly improved torsional rigidity and ultimate torque (FIGS. 5A and 5B). Compared with unfractured normal bone, BMP-2 coated allografts restored about 50% of the strength of bone at 7 weeks post-implantation.

### Activation of TGF-β Signaling Pathway by PLGA Coating and Antagonism of TGFβ and BMP-2

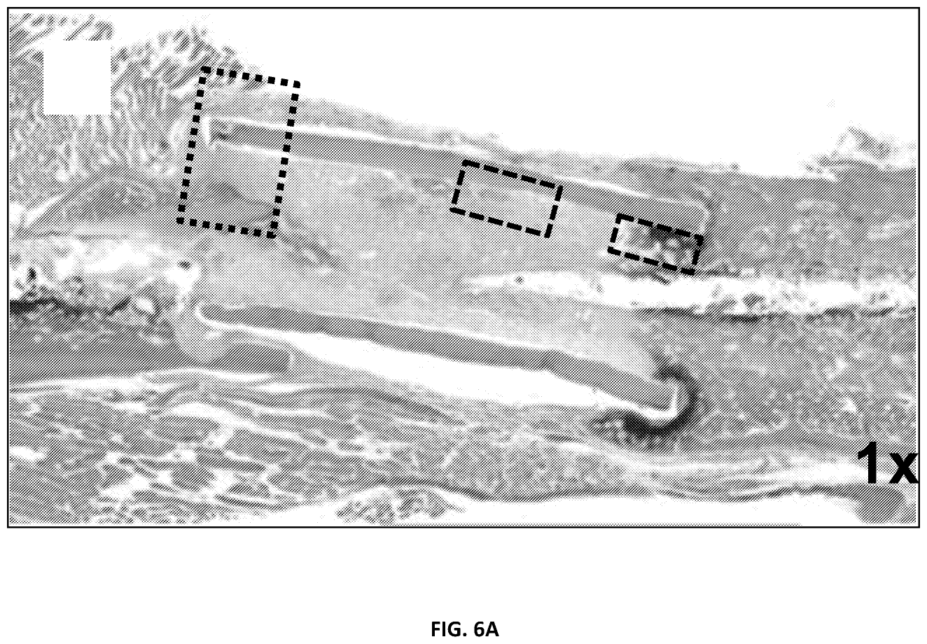

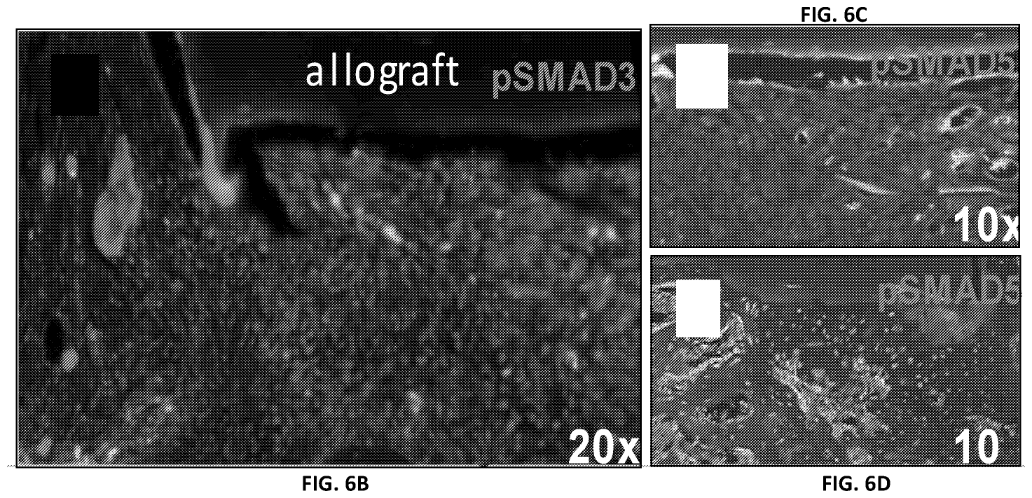

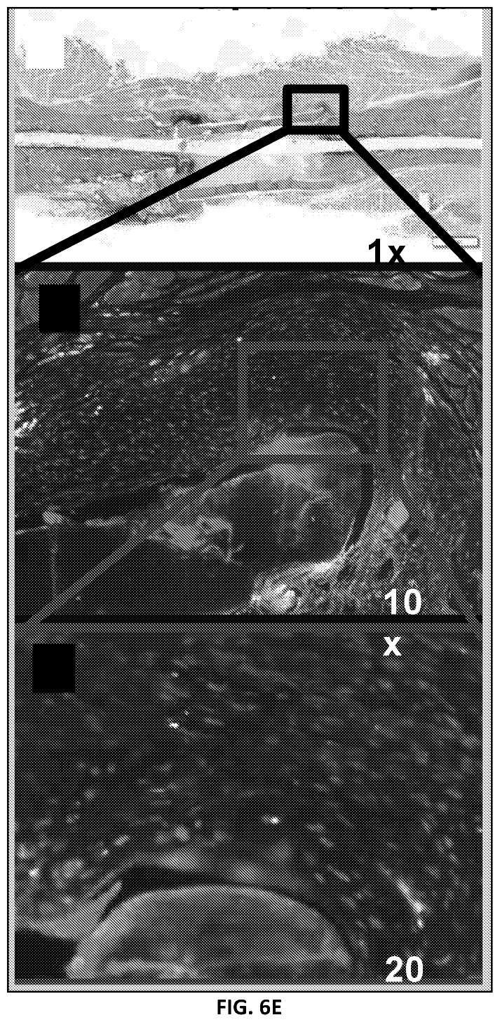

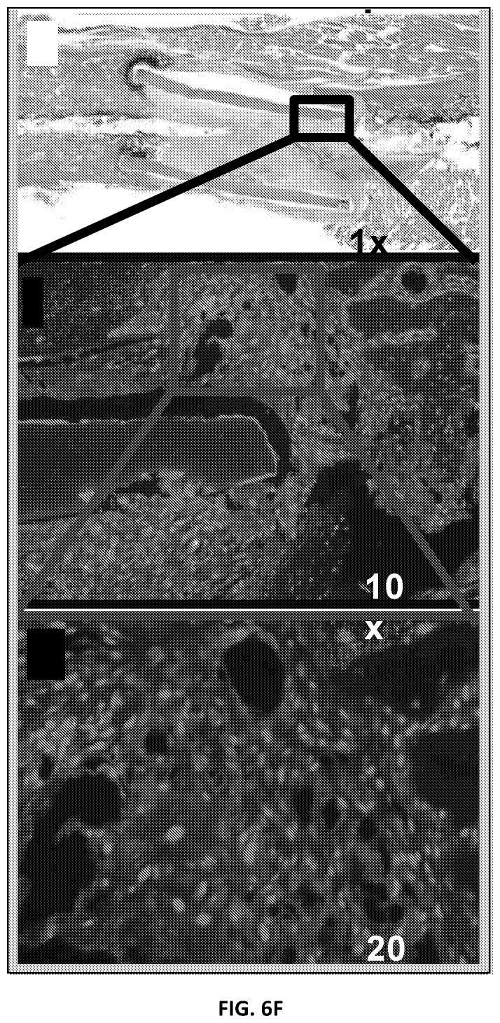

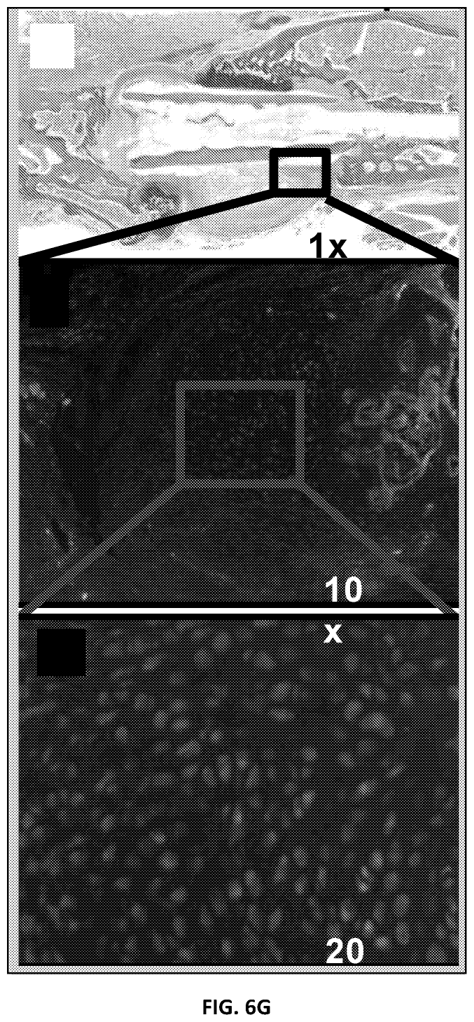

To further understand the potential mechanism of fibrosis induced at the site of allograft repair, immunofluorescence staining of pSMAD 3 and 5 were conducted in allograft samples. High level of pSMAD3 was found in fibrotic tissue and immature cartilage along the surface of allografts and at the cortical bone junctions (FIGS. 6A and 6B), indicating a role of TGF-β signaling in fibrotic tissue formation during allograft healing. In contrast, pSMAD5 was negative in fibrotic tissue but strongly positive in chondrocytes and bone cells (FIGS. 6C and 6D). Comparison of pSMAD3 level was conducted in three groups of allograft samples. While all three groups showed increased pSMAD3 level in fibrotic tissue, PLGA coated allograft demonstrated significant higher levels of pSMAD3 in fibrotic tissue adjacent to bone. With increased bone formation, BMP peptide coated allografts showed reduced level of pSMAD3 in tissue, indicating the antagonism of the BMP and TGF-β signaling pathway during allograft healing.

To create an improved structural bone allograft for repair and reconstruction of segmental bone defect, a reproducible method was devised to endow allograft with growth factor releasing property through polymer-mediated electrospray deposition. By controlling the polymer degradation, the release profile of the endowed peptide can be tailored to facilitate graft healing and incorporation. Further implantation of the BMP-2 peptide releasing allografts in a murine segmental femoral bone defect model showed significantly improved allograft healing as evidenced by enhanced bone formation on allograft surface and markedly suppressed fibrotic tissue formation, leading to improved integration of the grafts to host bone tissue.

While electrospray-mediated polymer deposition has been used in fabrication of biomaterials in the form of nano/micro particles and thin films, the approach has not been previously used to coat bone allograft for drug delivery and surface modification. It is shown hereinabove that this technique can be adopted to modify bone allograft by creating a bioactive surface, optionally ranging from about 1 μm to several hundred microns or more. The surface morphology as well as the drug loading and release can be easily adjusted by control the molecular structure, chemical and physical properties as well as the degradation rate of the polymers. Herein, a PLGA co-polymer (poly(DL-lactide-co-glycolide 50:50) with the incorporation of an osteogenic BMP-2 peptide was used for electrospray deposition and spin coating. Due to the hydrophobic nature of the peptide, the release of the BMP-2 peptide from the allograft was found to be largely dependent on the degradation rate of the PLGA, which can be tailored by varying the ratio of lactide and glycolide monomers. Nearly all loaded peptide was released during the first 4 weeks at chondrogenic and osteogenic phase of allograft healing, leading to significantly induction of bone and cartilage formation near the allograft surface.

Compared to autograft healing which leads to extensive bone formation along the bone graft surface, the inert allograft surface is often associated with fibrotic tissue formation and extremely poor mineralization of the allograft surface (Wang, et al. (2018) Biomaterials 182:279-88; Zhang, et al. (2005) J. Bone Miner. Res., 20:2124-37; Xie, et al. (2007) Tissue Eng., 13:435-45; Zhang, et al. (2008) Clin. Orthop. Relat. Res., 466:1777-87). Proper modification of the allograft surface will significantly enhance the mineralization and remodeling of the allograft, facilitating revitalization of the allografted bone. The effectiveness of the allograft coating was demonstrated in the current MicroCT analyses (FIG. 3). In addition to enhanced bone formation, it was found that more bone was formed near the allograft surface and some completely integrated along bone surface in BMP-2 peptide coated allograft samples, indicating that coating and modification of graft surface is effective in stimulating the allograft surface mineralization, enhancing the osseointegration and biomechanical function of the grafted bone.

While significantly enhanced healing was observed in BMP-2 peptide coated samples, bone formation along the surface of allograft was found to be uneven. This could be attributed to the uneven degradation of the polymers and insufficient osteoinductivity of the BMP-2 peptide. In addition to modulating the timing of the BMP-2 peptide release, a number of novel osteogenic and angiogenic peptides (Ciccarelli, et al. (2006) Circulation 114(s18):251 Abstract 1323; Rahman, et al. (2016) Circ. Res., 118:957-69; Wang, et al. (2017) Regenerative Biomater., 4:191-206; Bab, et al. (1992) EMBO J., 11:1867-73; Gabet, et al. (2004) Bone 35:65-73) can be used in the current application to enhance healing and graft integration. The versatility of the electrospray-mediated approach, which allows layer-by-layer deposition of multiple polymer components on bone allograft surface, will facilitate production of novel off-the-shelf allografts capable of releasing multiple growth factors/peptides at multiple time scales for repair and reconstruction.

Bone allografts with or without coatings could trigger a host immune response that leads to chronic inflammation and fibrotic tissue formation (Bannister, et al. (2008) J. Periodontology 79:1116-20; Nuss, et al. (2008) Open Orthopaedics J., 2:66-78). Transforming growth factor-β (TGF-β), especially isoform 1, is expressed by macrophages and is known to promote fibrosis in many cells and organs, including the lungs, kidneys, liver, heart, and skin. TGF-β signaling is ubiquitously activated in fibrotic diseases and is sufficient and required for the induction of fibrosis (Kajdaniuk, et al. (2013) Endokrynol. Pol., 64:384-96; Meng, et al. (2016) Nat. Rev. Nephrology 12:325-38). Multiple strategies to interfere at different steps of TGF-β signaling have shown promising anti-fibrotic effects in preclinical models (Gyorfi, et al. (2018) Matrix Biol., 68-69:8-27; Walton, et al. (2017) Front. Pharmacol., 8:461). A number of reagents targeting TGF-β signaling have either been approved or in advanced clinical development to suppress scar and fibrotic tissue formation in vital organs. Herein, it was found that TGF-β signaling as indicated by p-SMAD3 expression is markedly enhanced in fibrotic tissue in allograft samples, with significantly stronger and more staining in allografts coated with PLGA, indicating a role of TGF-β in polymer induced fibrotic tissue formation at the healing site. Remarkably, coating of BMP-2 peptide with the same PLGA as vehicle, the fibrotic response was markedly tempered, accompanied by significant reduction of the p-Smad3 staining in allograft samples. This data strongly indicates the antagonism between BMP and TGF-β signaling pathway.

TGF-β/BMP superfamily of ligands are known to interact with their respective receptors ALK1/2/3/6 or ALK4/5/7, the former being mostly BMPs and the latter being mostly activins and TGF-βs. Signaling pathway of BMPs activates R-SMADs 1, 5, and 8, whereas the signaling pathway of TGF-βs activates R-SMADs 2 and 3. Both pathways converge at the common transcription factor SMAD4, which may lead to synergistic or opposing effects depending on the stimulation strategies (Wu, et al. (2016) Bone Res., 4:16009). While both BMPs and TGF-βs play important roles in bone development and remodeling, a significant amount of literature also demonstrates a reciprocal and opposing effects of BMP and TGF-β signaling on osteoblast and chondrocyte differentiation (Mizuno, et al. (2009) FEBS Lett., 583:2263-8; Maeda, et al. (2004) EMBO J., 23:552-63; Li, et al. (2006) J. Bone Miner. Res., 21:4-16; Wang, et al. (2019) Orthopaedic Research Society Annual meeting 2019:0235). When activin/TGF-0 signaling is inhibited in vivo through genetic or pharmacologic approaches, bone formation rate and bone mass increase (Edwards, et al. (2010) J. Bone Miner. Res., 25:2419-26; Qiu, et al. (2010) Nat. Cell Biol., 12:224-34; Koncarevic, et al. (2010) Endocrinology 151:4289-300; Ruckle, et al. (2009) J. Bone Miner. Res., 24:744-52; Pearsall, et al. (2008) Proc. Natl. Acad. Sci., 105:7082-7). Inhibition of TGF-β signaling also enhances osteoblast differentiation of bone marrow stromal cells and preosteoblastic MC3T3-E1 cells, and further potentiates the effects of BMP-2 (Maeda, et al. (2004) EMBO J., 23:552-63; Takeuchi, et al. (2010) PLoS One 5:e9870). In view of the current study, the antagonism between these two pathways can be exploited for therapeutic purposes (Hudnall, et al. (2016) J. Amer. Osteopathic Assn., 116:452-61).

Herein, a reproducible method was developed to coat and endow drug releasing properties on cortical allograft via polymer-mediated electrospray deposition. The modified allografts demonstrated sustained release of BMP peptide and improved graft healing when used to repair segmental bone defects. While PLGA coated allografts showed enhanced fibrotic tissue formation associated with increased TGFβ signaling, inclusion of BMP peptide in PLGA coating antagonize the fibrotic tissue formation by significantly reducing TGF-β signaling, indicating that controlled antagonism between TGF-β and BMP-2 signaling is a viable therapeutic strategy to enhance allograft repair and incorporation. The present system allows for the delivery of multiple osteogenic and angiogenic factors through allograft coating and surface modification.

### Example 2

Both biomaterials and allografts can trigger a host immune reaction that leads to fibrotic tissue formation and implant failure (Nuss, et al. (2008) Open Orthopaedics J., 2:66-78; Anderson, et al. (2008) Seminars Immunology 20:86-100; Klopfleisch, et al. (2017) J. Biomed. Mater. Res., 105:927-940). Macrophages and TGF-β signaling may play a role in this process (Hernandez-Pando, et al. (2000) Immunology 100:352-358; Khouw, et al. (1999) Biomaterials 20:1815-1822; Rolfe, B. E. (2011) Regenerative Me. Tissue Engineering 26:551-568). TGF-β signaling is ubiquitously activated in fibrotic diseases and is sufficient and required for the induction of fibrosis (Kajdaniuk, et al. (2013) Endokrynol. Pol., 64:384-396; Meng, et al. (2016) Nature Rev. Nephrology 12:325-338). Inhibition of TGF-β signaling through genetic or pharmacologic approaches can increase bone formation rate and bone mass (Edwards, et al. (2010) J. Bone Miner. Res., 25:2419-2426; Qiu, et al. (2010) Nature cell biol., 12:224-234; Koncarevic, et al. (2010) Endocrinology 151:4289-4300; Ruckle, et al. (2009) J. Bone Miner. Res., 24:744-752; Pearsall, et al. (2008) Proc. Natl. Acad. Sci., 105:7082-7087). Inhibition of TGF-β signaling can also enhance osteoblast differentiation of bone marrow stromal cells and preosteoblastic MC3T3-E1 cells, and further potentiate the effects of BMP-2 (Maeda et al. (2004) EMBO J., 23:552-563; Takeuchi, et al. (2010) PLoS One 5:e9870).

A reciprocal and opposing relationship between TGF-β signaling and BMP signaling exists in the control of the differentiation of osteoblasts and chondrocytes (Spinella-Jaegle, et al. (2001) Bone 29:323-330; Mizuno, et al. (2009) FEBS Lett., 583:2263-2268; Maeda, et al. (2004) EMBO J., 23:552-563; Li, et al. (2006) J. Bone Miner. Res., 21:4-16; Li, et al. (2005) Front. Biosci., 10:681-688; Hudnall, et al. (2016) J. Amer. Osteopathic Assoc., 116:452-461). However, the reciprocal regulation between TGF-β and BMP-2 signaling has not yet been explored in bone healing and implant-associated fibrosis. Herein, it is shown that delivery of a BMP-2 mimicking peptide and an anti-fibrotic TGF-β signaling inhibitor via allograft surface coating on promoting bone formation and inhibiting fibrotic response in defect repair and reconstruction. Controlled antagonism of BMP-2 and TGF-β signaling promotes osteogenic BMP-2 signaling and suppresses fibrogenic TGF-β signaling leading to reduced fibrotic response, enhanced osseointegration, and improved structural bone allograft healing.

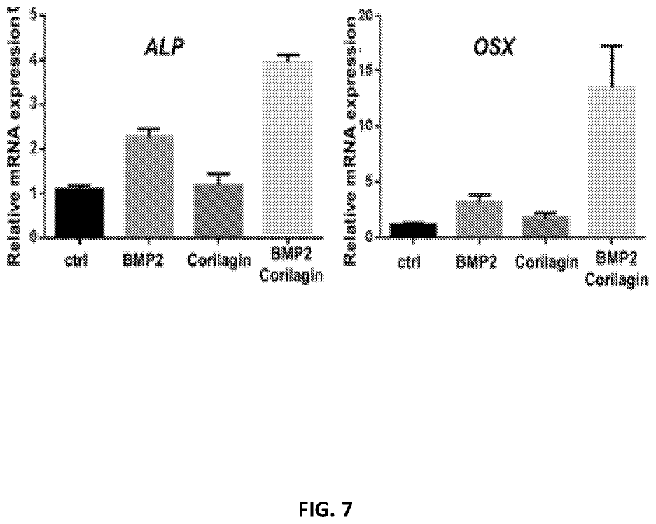

To test the potential reciprocal regulation of both BMP and TGF-β signaling in repair, periosteal progenitor cells were isolated from autograft periosteum (Wang, et al. (2010) Am. J. Pathol., 177:3100-3111; Huang, et al. (2014) Mol. Ther., 22:430-439; Huang, et al. (2014) PLoS One, 9:e100079). The osteogenic differentiation of BMP-2 (50 μg/ml) in combination with TGF-β inhibitors was examined. Corilagin (1 μm) potentiated the effects of BMP-2 on osteoblastic differentiation as evidenced by increased alkaline phosphatase (ALP) staining and enhanced expression of ALP and osterix (Osx) in 10-day cultures (FIG. 7).

Corilagin is an inhibitor of TGF-01 signaling and strongly inhibits Tgfbrl/ALK5 kinase activity without inhibiting the BMP type I receptors ALK2, ALK3, and ALK6 (Wei, et al. (2017) J. Clin. Invest., 127:3675-3688; Jia, et al. (2013) BMC Complemen. Altern. Med., 13:33; Duan, et al. (2019) J. Biol. Chem., 294:8490-8504). Corilagin is a medicinal herbal agent discovered in medicinal plants such as Phyllanthus species and has anti-tumor, anti-inflammatory, and antioxidative effects in animal models (Li, et al. (2018) Biomed. Pharmacother., 99:43-50). Corilagin also inhibits TGF-β-dependent epithelial-mesenchymal transition (EMT) and attenuates fibrosis in lung in a mouse model (Wei, et al. (2017) J. Clin. Invest., 127:3675-3688). Corilagin also shows a low level of toxicity toward normal cells and tissues. As seen in FIG. 7, corilagin showed strong synergism with BMP-2.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

1: A method of preparing a coated bone graft, said method comprising electrospraying a composition comprising a polymer and a therapeutic agent onto the surface of the bone graft, thereby preparing said coated bone graft.

2: The method of claim 1, wherein said therapeutic agent is selected from the group consisting of bone stimulating agents, anti-fibrotic agents, antimicrobials, anti-inflammatory agents, and pro-angiogenesis agents.

3: The method of claim 1, wherein said therapeutic agent is a bone stimulating agent.

4: The method of claim 3, wherein said bone stimulating agent is bone morphogenetic protein 2 (BMP-2) or a fragment thereof.

5: The method of claim 4, wherein said BMP-2 fragment comprises SEQ ID NO: 1.

6: The method of claim 1, wherein said polymer is a hydrophobic polymer.

7: The method of claim 6, wherein said polymer is poly(lactide-co-glycolide).

8: The method of claim 1, wherein said composition comprises a bone stimulating agent and an anti-fibrotic agent.

9: The method of claim 8, wherein said bone stimulating agent is bone morphogenetic protein 2 (BMP-2) or a fragment thereof and said anti-fibrotic agent is corilagen.

10: The method of claim 1, wherein said method comprises i) electrospraying a first composition comprising a polymer and, optionally, a therapeutic agent onto the surface of the bone graft, and ii) electrospraying a second composition comprising a polymer and, optionally, a therapeutic agent onto the surface of the coating produced by step i).

11: The method of claim 1, wherein the coating of the coated bone graft is about 1 μm to about 1 mm thick.

12: The method of claim 1, further comprising freeze drying and/or lyophilizing the synthesized coated bone graft.

13: The method of claim 1, further comprising mineralizing the synthesized coated bone graft.

14: The method of claim 1, wherein said bone graft is a bone allograft.

15: A coated bone graft prepared by the method of claim 1.

16: A coated bone graft comprising a bone graft and an electrosprayed coating on the surface of the bone graft, wherein the electrosprayed coating comprises a polymer and a therapeutic agent.

17: The coated bone graft of claim 16, wherein said therapeutic agent is selected from the group consisting of bone stimulating agents, anti-fibrotic agents, antimicrobials, anti-inflammatory agents, and pro-angiogenesis agents.

18: The coated bone graft of claim 16, wherein said therapeutic agent is a bone stimulating agent.

19: The coated bone graft of claim 18, wherein said bone stimulating agent is bone morphogenetic protein 2 (BMP-2) or a fragment thereof.

20: The coated bone graft of claim 19, wherein said BMP-2 fragment comprises SEQ ID NO: 1.

21: The coated bone graft of claim 16, wherein said polymer is a hydrophobic polymer.

22: The coated bone graft of claim 21, wherein said polymer is poly(lactide-co-glycolide).

23: The coated bone graft of claim 16, wherein said coating comprises a bone stimulating agent and an anti-fibrotic agent.

24: The coated bone graft of claim 23, wherein said bone stimulating agent is bone morphogenetic protein 2 (BMP-2) or a fragment thereof and said anti-fibrotic agent is corilagen.

25: The coated bone graft of claim 16, wherein said coating comprises more than one layer.

26: The coated bone graft of claim 16, wherein the coating of the coated bone graft is about 1 μm to about 1 mm thick.

27: A method for treating a bone defect in a subject, said method comprising implanting the coated bone graft of claim 16 into the subject.

28: The method of claim 27, wherein said bone graft is a bone allograft.
